(19) United States
(12) Patent Application Publication
(10) Pub. No.: US 2010/0021895 A1
(43) Pub. Date: Jan. 28, 2010

(54) Vectors for Inducing Homozygous Mutations and Methods of Using Same

(76) Inventor: H. Earl Ruley, Nashville, TN (US)

Correspondence Address: Zirus, Inc.;C/O Ballard Spahr LLP, 999 Peachtree Street, Suite 1000, Atlanta, GA, 30309-3915, US

(21) Appl. No.: 12/373,410

(22) PCT Filed: Jul. 12, 2007
(86) PCT No.: PCT/US07/15888

§ 371 Date: Jul. 30, 2009

Related U.S. Application Data
(60) Provisional application No. 60/830,219, filed on Jul. 12, 2006.

(51) Int. Cl. C12Q 1/68; C12N 15/63; C12Q 1/02

(52) U.S. Cl. 435/6; 435/320.1; 435/29

(57) ABSTRACT

The present invention provides vectors for inducing homozygous mutations in cells. Also provided are cells and populations of cells comprising a vector of the present invention. Further provided are methods of identifying cells with homozygous mutations. Also provided are methods of identifying agents that increase the frequency of homozygous mutations in cells. The present invention also provides methods of identifying a gene that is responsible for a recessive genetic trait.

### CROSS-REFERENCE TO RELATED APPLICATIONS

This application claims benefit of U.S. Provisional Application No. 60/830,219, filed Jul. 12, 2006, herein incorporated by reference in its entirety.

### BACKGROUND OF THE INVENTION

The number and diversity of genes identified by the mammalian genome projects suggests that considerable biology remains to be characterized on a molecular level and has provided the impetus for developing genome-wide strategies to characterize gene functions important in normal and disease processes. Tagged sequence mutagenesis uses gene entrapment vectors to disrupt genes in cultured cells combined with rapid, DNA sequence-based screens to characterize the disrupted genes at the nucleotide level. The approach has been widely used to disrupt genes in mouse embryonic stem (ES) cells (1-3) and to a far lesser extent to identify genes responsible for recessive phenotypes in somatic cells (4-8).

Mutagenesis of mammalian cells is hindered by the fact that the normal genome is diploid and consequently, most entrapment mutations are recessive. The problem is circumvented by gene-based studies in ES cells where selected mutations can be transmitted through the mouse germline and subsequently bred to a homozygous state. However, gene inactivation in somatic cells requires pre-existing hemizygosity or spontaneous loss of heterozygosity; thus, even with strategies to enhance the recovery of loss-of-function mutations (4,5,7,9,10), entrapment mutagenesis has seen only limited use in phenotype-driven screens.

Mammalian cells heterozygous at a given locus undergo spontaneous conversion to a homozygous state by loss of heterozygosity (LOH) at frequencies of about 10⁻⁵ per cell (11-14). Homozygous mutants can be selected based on phenotypes caused by gene dosage effects. For example, mutations involving the insertion of a neomycin resistance gene (Neo) may be converted to a homozygous state simply by selecting for clones that survive in higher concentrations of G418 (15). Levels of neomycin resistance correlate with levels of Neo gene expression (16). Mitotic recombination—which leads to LOH and doubles the number of Neo genes per cell—appears to be the preferred mechanism by which moderately resistant cells spontaneously acquire resistance to higher antibiotic concentrations (17). However, unlike targeted mutations, LOH has not been reliably achieved with mutations induced by gene entrapment. A major problem stems from variations in Neo gene expression that can result, for example, when the entrapment cassette is expressed from different cellular promoters. Thus, there is a need for methods that can reliably achieve loss of heterozygosity.

### BRIEF DESCRIPTION OF THE FIGURES

FIG. 1A shows structures of the GTR retrovirus gene trap vectors. Expression of an intron-containing Neo gene (5′Neo+3′Neo) carried by the GTR1.0 poly(A) trap vector selects for inserts in which the Neo gene, expressed from the RNA polymerase 2 promoter (Pol2), splices to downstream exons of cellular genes, Transcripts of occupied cellular genes splice to a 3′ exon [consisting of the 3′ end of a puromycin resistance gene (3′ Puro), an internal ribosome entry site (IRES), a lacZ reporter and a polyadenylation site (PA)], disrupting their expression. A wild type loxP site (loxP, left of 3′ Puro) and mutant loxP sites (lox 5171, on either side of the 3′ Neo exon) allow the body of the provirus to be replaced by other sequences by Cre-mediated cassette exchange. An RNA instability sequence (MI, flash symbol) increases the specificity of gene entrapment by reducing the levels of unspliced Neo transcripts. The positions of the provirus long terminal repeats (3′ and 5′ LTRs) are also indicated. Viruses lacking either the message instability sequence (pGTR1.3) or the lox 5171 in the Neo intron (GTR1.2) or both elements (GTR1.1) have also been constructed. GTR1.4-1.7 are identical to GTR1.0-1.3 except they contain an enhanced green fluorescence (EGFP) reporter instead of lacZ. GTR2.0-2.3 are identical to GTR1.0-1.3 except Neo is expressed from the PGK promoter. Some elements are not drawn to scale to enhance clarity.

FIG. 1B shows direct cloning of 3′ RACE products. Gene entrapment by GTR vectors generates clones in which the Neo gene (white boxes) is expressed from transcripts that splice to downstream exons of cellular genes (black boxes). The intron and NotI endonuclease cleavage site in the Neo coding sequence ensure that only recombinant plasmids that contain cDNA inserts amplified from spliced Neo-cell fusion transcripts can give rise to kanamycin-resistant E. coli.

FIG. 2A shows tagged sequence mutagenesis with the GTR gene trap vectors. 974 vector-fusion transcripts cloned by 3′RACE matched sequences in the EST, mouse genome (MM) and NT databases as shown (a). Matches corresponding to cellular transcription units (unigene) based on the genome sequence annotation are also indicated. (b) The positions of GTR inserts in cellular genes as deduced from the sequence of 3′RACE products. The majority of inserts (187) spliced to the last exon of cellular genes; of these, 138 contained multiple annotated exons and 49 contained two exons.

FIG. 3 shows the loss of occupied gene expression in homozygous mutant cells and tissues. Pfdn1 expression (a, top panel) in embryonic fibroblast cells from wild-type (lane 1), homozygous mutant (lane 2) and heterozygous (lane 3) fibroblasts was analyzed by Northern blot analysis. The blot was stripped and probed with a GAPDH sequence as a loading control (lower panel). Cradd protein expression (b, top panel) in primary speen cells from wild type (lane 1) and homozygous mutant mice (lame 2) was assessed by western blot analysis. As a loading control, the blots were stripped and analyzed using an anti-β-actin antibody (bottom panel). Dymeclin expression (c, top panel) in liver tissue from wild-type (lane 1) and homozygous mutant mice (lane 2) was analyzed by northern blot analysis. Hybridization to a GAPDH probe (lower panel) provides a loading control.

FIG. 4 shows LOH at entrapment loci following selection in 2.0 mg/ml G418. DNAs from the parental ES cells (lane 1), heterozygous mutant entrapment clones (lane 2) and clones isolated following selection in 2.0 mg/ml G418 (lanes 3-7) were genotyped by either Southern blot hybridization (j, l) or PCR (a-I, k) using gene-specific probes and primers. The mutant clones contained entrapment vectors inserted in the following genes: Col12a1 (a) Rbm4 (b), IL8-Ra (c), 1810059C17Rik (d), Cradd (e), Ep400 (f), unknown (g), D130017N08Rik (h), Hesx1 (i), 1810030N24Rik (j), Cnr2 (k), and Xrcc5 (l).

FIG. 5 shows the frequency of presumptive LOH at sites throughout the genome increases with distance from the centromere. 37 ES cell clones, each containing a single GTR1.3 gene trap vector, were placed in media containing 2.0 mg/ml and 0.3 mg/ml G418. The frequency of colony formation (Presumptive LOH) in the higher concentration of G418 (normalized to the number of colonies at the lower concentration) is plotted against the distance of each mutation from the centromere. The average values for 3 independent experiments are plotted for all 37 entrapment loci (a) and for all 8 inserts located on chromosome 4 (b). Linear regression analyses of the two groups produced R-squared values of 0.54 and 0.78, respectively. The standard deviations, which were 10-60% of the average values, have been omitted for clarity.

FIG. 6 shows Loss of Xrcc5 expression in homozygous entrapment clones. (a) RNA was isolated from the parental AC1 ES cells (lane 1), the heterozygous Xrcc5 entrapment mutant before selection in 2.0 mg/ml G418 (lane 2) and clones isolated by selection in 2.0 G418 (lanes 3-6) and Northern blot analysis was performed using Xrcc5 (downstream of exon 1 cloned by 3′RACE) and β-actin specific probes (top and bottom panels, respectively). Neo-Xrcc5 fusion transcripts in heterozygous and homozygous mutant cells are generated by splicing of the Neo sequences to exon 2 of the Xrcc5 gene. (b) Radiation sensitivity of Xrcc5 heterozygous and homozygous mutant cells. Parental ES cells (1), heterozygous Xrcc5 entrapment mutant (2) homozygous Xrcc5 entrapment mutants (3, 5, 6) and a control Xrcc6-deficient Chinese hamster ovary cell line (4) were exposed to increasing doses of γ-irradiation, and cell survival was measured in a clonogenic assay.

FIG. 7 shows GTR vector construction. (A) GTR vectors were assembled by joining the plasmid vector backbone to the 5′ and 3′ entrapment, cassettes. (B) Structure of the 3′ entrapment cassette. (C) Intron sequence (lower case) inserted into the neomycin resistance gene (upper case). A NotI site inserted into the Neo gene and lox5171 site in the intron are indicated. (D) Structure of the 3′ entrapment cassette. See Example I for details.

FIG. 8 shows distribution of entrapment mutations throughout the murine genome. Stars represent the approximate locations of the 37 clones with GTR1.3 retroviral vector inserts on murine chromosomes 1-12, 14, 15, 17-19, (dark gray) and the 5 clones in which LOH was molecularly verified (FIG. 3) with GTR2.3 retroviral vector inserts on murine chromosomes 2, 4, 7, and 12 (light gray).

FIG. 9 shows the distribution of entrapment mutations in the murine genome. Stars represent the locations of GTR1.3 retroviral vector inserts in 53 clones on murine chromosomes 1-15, and 17-19. The centromere for each chromosome is positioned at the top of the idiogram.

FIG. 10 shows that limited carcinogen exposure enhances the survival of mutant ES cells in media containing 2.0 mg/ml G418. ES cells heterozygous for an entrapment mutation in Xrcc5 were selected in high G418 directly (a) or following treatment for 4 hours with 0.5 mM methyl-nitrosourea (b), 0.25 mM hydroxyurea (c), or 100 ng/ml diepoxybutane (d). After 12 days in selection, colonies were washed with PBS and stained with crystal violet.

FIG. 11 shows that LOH occurs at entrapment loci in clones selected in high G418. DNAs from the parental (AC1) ES cells (Lane 1), heterozygous mutant entrapment clones (lane 2), and clones isolated in high G418 without treatment (lanes 3-6) and following treatment with methyl-nitrosourea (Lanes 7-10) or hydroxyurea (lanes 11-14) were genotyped either by Southern blot hybridization (a, d) or by PCR (b-d) using gene-specific probes and primers. The mutant clones contained entrapment vectors inserted in the 1810030N24Rik (a), Hesx1 (b), IL8Ra (c), Cradd (d), and Xrcc5 (e) genes. Following carcinogen treatment and selection in 2.0 mg/ml G418, LOH was observed in 72, 24, 36, 24, and 232 independent clones, respectively.

FIG. 12 shows the effect of chromosome position on chemically-induced survival in high G418. 53 ES cell clones each containing a single gene trap vector were treated for 4 hours with 0.5 mM methyl-nitrosourea (squares), 0.25 mM hydroxyurea (triangles) or were untreated (circles) and then placed in media containing 2.0 mg/ml G418. The frequency of colony formation (presumptive LOH) for each clone is plotted against the location of each entrapment mutation (distance from the centromere). Linear regression analysis of all clones in aggregate (a) produced R2 values for untreated, HU- and MNU-treated cells of 0.62, 0.53, and 0.08, respectively. R2 values for all clones with mutations on chromosome 4 (b) were 0.68, 0.65, and 0.11 for untreated, HU- and MNU-treated cells, respectively.

FIG. 13 shows sensitivity of embryo-derived stem cells to various carcinogenic agents. Survival of the parental embryo-derived stem cells was determined following 4-hour exposure to the indicated agents. Experiments were repeated 10 times (each line represents an independent experiment). Percent survival refers to the percentage of cells capable of forming viable colonies as compared to untreated cells. Arrows indicate the concentration of each agent chosen for LOH studies.

FIG. 14 shows a time course of carcinogen-induced LOH. AC1 ES cells were treated for 4 hours with 0.5 mM methyl-nitrosourea (open circles), 0.25 mM hydroxyurea (squares) or were untreated (solid circles) and then placed in media containing 2.0 mg/ml G418 at the indicated times thereafter. The percent of colony forming cells is plotted for each time point.

### SUMMARY OF INVENTION

The present invention provides vectors for inducing homozygous mutations in cells. Also provided are cells and populations of cells comprising a vector of the present invention. Further provided are methods of identifying cells with homozygous mutations. Also provided are methods of identifying agents that increase the frequency of homozygous mutations. The present invention also provides methods of identifying a gene that is responsible for a recessive genetic trait.

### DETAILED DESCRIPTION OF THE INVENTION

Before the present methods and systems are disclosed and described, it is to be understood that this invention is not limited to specific synthetic methods, specific components, or to particular compositions, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The present invention may be understood more readily by reference to the following detailed description of preferred embodiments of the invention and the Examples included therein and to the Figures and their previous and following description.

The present invention shows that entrapment mutations generated by a poly(A) trap (18-20) can be reproducibly converted to homozygosity, when the heterozygous mutant cells express similar, moderate levels of neomycin resistance. New poly(A) trap vectors were developed for this purpose in which gene entrapment selects for inserted Neo sequences that splice to the 3′ ends of cellular genes. The vectors have additional features that facilitate the identification of disrupted genes and that allow genes and chromosomes tagged by gene entrapment to be engineered by DNA site-specific recombinases (21-26). The vectors are suitable for large-scale mutagenesis of mouse ES cells, and the present invention shows that most mutations selected from a stem cell library can be converted to a homozygous state following selection for higher levels of drug resistance. The ease and efficiency of obtaining homozygous entrapment mutations (i) facilitates genetic studies of gene function in cultured cells, (i) permits genome-wide studies of recombination events that result in LOH and mediate a type of chromosomal instability important in carcinogenesis, and (iii) provides new strategies for phenotype-driven mutagenesis screens in mammalian cells.

The present invention provides a retroviral poly(A) trap vector comprising a nucleotide sequence between a 5′ LTR and a 3′ LTR, wherein said nucleotide sequence comprises 1) an intron containing nucleic acid encoding a first selective marker operably linked to a promoter, 2) site specific recombinase sites, and 3) a 3′ exon comprising a nucleic acid encoding the 3′ segment of a second selective marker, an internal ribosome entry site (IRES), a nucleic acid encoding a reporter protein and a polyadenylation site. The present invention also provides cells comprising a retroviral poly(A) trap vector of this invention. Further provided are cells wherein the retroviral vector is integrated into the genome of the cell. Such integration can be transient or stably transmitted through the germline. A cell comprising a retroviral vector of the present invention can be an in vitro, ex vivo or an in vivo cell.

The retroviral vector according to the present invention can be based on any retrovirus. Therefore, the poly(A) trap vectors of the present invention can comprise any retroviral genome comprising a heterologous nucleotide sequence that is inserted between the 5′ LTR and the 3′LTR of the retroviral genome. Thus, sequence(s) that are normally found between the 5′ LTR and the 3′ LTR of a retroviral genome can be deleted/replaced with the heterologous sequences mentioned herein. As mentioned above, these heterologous sequences include, but are not limited to, an intron containing nucleic acid encoding a first selective marker operably linked to a promoter, 2) site specific recombinase sites and 3) a 3′ exon comprising a nucleic acid encoding the 3′ segment of a second selective marker, an internal ribosome entry site (IRES), a nucleic acid encoding a reporter protein and a polyadenylation site. According to the present invention, the retroviral poly(A) trap vector may comprise up to about 7 kilo base pair (kbp) of heterologous sequences, up to about 6 kbp, up to about 5 kbp, up to about 4 kbp, up to about 2 kbp, up to about 1 kbp and up to about 0.5 kbp heterologous sequences. As utilized herein, “heterologous” means any combination of nucleic acid sequences that is not normally found associated in nature.

The retroviral poly(A) trap vectors of the present invention can be based on any retroviral genome, including but not limited to, a murine leukemia virus (MLV) such as, for example, moloney murine leukemia virus (MMLV) (see GenBank Accession No. AF033811 for nucleotide sequence (SEQ ID NO: 1)), Akv-murine leukemia virus (Akv-MLV) (see GenBank Accession No. J01998 (SEQ ID NO: 2)), Abelson murine leukemia virus (see GenBank Accession No. AF033812 for nucleotide sequence (SEQ ID NO: 3)), Friend murine leukemia virus (see GenBank Accession No. Z11128 for nucleotide sequence (SEQ ID NO: 4)), Rauscher murine leukemia virus (see GenBank Accession No. U94692 for nucleotide sequence (SEQ ID NO: 5)), murine type C retrovirus (see GenBank Accession No. X94150 for nucleotide sequence (SEQ ID NO: 6)) or SL-3-3-murine leukemia virus (SL3-3-MLV) (see GenBank Accession No. AF169256 for nucleotide sequence (SEQ ID NO: 7)) or any retrovirus with a nucleotide sequence of 80% homology or greater to any murine leukemia virus.

The vectors can also be based on lentiviral genomes or any retrovirus with a nucleotide sequence of 80% homology or greater to any lentiviral genome. Such genomes include, but are not limited to, a primate lentivirus (see [U.S. Pat. No. 5,665,577]), a human immunodeficiency virus (HIV) (see GenBank Accession No. NC_{—}001802 (SEQ ID NO: 8) and GenBank Accession No. NC_{—}001722 (SEQ ID NO: 9)) (J. Reiser et al., Proc. Natl. Acad. Sci. USA, 93:15266-15271 (1996); and L. Naldini et al., Science, 272:263-267 (1996), a Visna/maedi virus (e.g., such as infect sheep) (see GenBank Accession No. NC_{—}001452 (SEQ ID NO: 10)), a feline immunodeficiency virus (FIV) (see GenBank Accession No. NC-001482 (SEQ ID NO: 11)) (Poeschla, E. M., et al., Nat. Medicine 4:354-357 (1998)), a bovine lentivirus (see GenBank Accession No. NC_{—}001413 (SEQ ID NO: 12)), a simian immunodeficiency virus (SIV) (see GenBank Accession No. NC_{—}004455 (SEQ ID NO: 13), GenBank Accession No. NC_{—}001549 (SEQ ID NO: 14) and GenBank Accession No. NC_{—}001870 (SEQ ID NO: 15)), an equine infectious anemia virus (EIAV) (see GenBank Accession No. NC_{—}001450 (SEQ ID NO: 16)), a Jembrana disease virus (see GenBank Accession No. NC_{—}001654 (SEQ ID NO: 17)), an ovine lentivirus (see GenBank Accession No. NC_{—}001511 (SEQ ID NO: 18)) and a caprine arthritis-encephalitis virus (CAEV) (see GenBank Accession No. NC_{—}001463 (SEQ ID NO: 19). The sequences, and the information set forth under the GenBank Accession Nos. set forth herein, for example, information on the location of the LTRs in the genome, and the location of other viral sequences are hereby incorporated by reference. Such vectors are useful for insertion into dividing and non-dividing cells. The vectors can also comprise hybrid retroviral sequences, for example, a vector can comprise a lentiviral sequence and a sequence from another retrovirus, such as a murine leukemia virus. It is also understood that the vectors of the present invention can also comprise a nucleic acid encoding a targeting polypeptide that allows delivery of the vector to specific cells or tissues. For example, the targeting polypeptide can be a ligand that binds a cell surface receptor. It would be routine for one of skill in the art to obtain a nucleic acid comprising a retroviral genome, identify the 3′ and 5′ LTR regions and insert heterologous sequences between these regions as described herein.

It is understood that as discussed herein the use of the terms “homology” and “identity” mean the same thing as similarity. Thus, for example, if the use of the word homology is used to refer to two sequences, it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids herein, is through defining the variants and derivatives in terms of homology to specific known sequences. In general, variants of nucleic acids and polypeptides herein disclosed typically have at least, about 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/bl2seq/bl2.html)), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

The retroviral vectors of the present invention can be constructed as described in the Examples. Furthermore, the retroviral vectors of the present invention can be used to selectively disrupt genes and then select cells homozygous for the mutations. Therefore, these vectors are capable of biallelic mutagenesis. In other words, these vectors can mutate both alleles of a gene.

The normal retroviral vector comprises two complete LTRs—a 5′ and 3′ LTR—both comprising subregions, namely the U3-, R- and U5-region. The U3 region incorporates all regulatory elements and/or promoters, which are responsible for the transcription and translation of the retroviral genome. Additionally, at the 5′ end of the U3-region the so-called inverted repeats (IR) are located. The IR are involved in the integration process of proviral DNA into the genome of a target cell. The R-region starts, per definition, with the transcription start codon and further comprises a polyadenylation signal. This polyadenylation signal, however, is only activated in the 3′LTR and thereby, marks the end point of a mature retroviral RNA transcript. It is assumed that, the U5 region of the LTR comprises one out of several packaging signals of the retroviral genome.

As utilized herein, a retroviral poly(A) trap vector is a vector that inserts a heterologous sequence, for example, a selectable marker, throughout the genome, wherein the heterologous sequence splices to 3′ distal exons of cellular genes. For example, the selectable marker can be an antibiotic resistance marker, for example neomycin, puromycin, hygromycin and the like.

As disclosed herein, the expression control sequences that drive expression of the heterologous sequence can include, but are not limited to, inducible and non-inducible promoters, enhancers, operators, sequences that destabilize RNA such as the Hepatitis Virus Delta and hammerhead ribozymes and 3′ untranslated sequences from c-fos and GM-CSF mRNAs, and other elements known to those skilled in the art. For example, the heterologous sequence may be placed under the control of a constitutive promoter or under an inducible promoter. Any expression sequence known in the art that is suitable for the expression of the heterologous sequence may be used with the retroviral vector of the present invention. Expression control sequences may include, but are not limited to, the cytomegalovirus (hCMV) immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors. Additional promoters include, the Gal4 promoter, the ADH promoter, PGK promoter, alkaline phosphatase promoter, an RNA polymerase II promoter, β-lactamase promoter and mammalian tissue specific promoters.

As mentioned above, the vectors of the present invention comprise site specific recombination sites. Site specific recombinases are enzymes that are present in some viruses and bacteria and have been characterized to have both endonuclease and ligase properties. These recombinases (along with associated proteins in some cases) recognize specific sequences of bases in DNA and exchange the DNA segments flanking those segments. To perform this exchange, the site-specific recombinase typically has the following four activities: (1) recognition of one or two specific DNA sequences; (2) cleavage of said DNA sequence or sequences; (3) DNA topoisomerase activity involved in strand exchange; and (4) DNA ligase activity to reseal the cleaved strands of DNA. Numerous recombinase systems are available to one of skill in the art. Perhaps the best studied of these are the Integrase/att system from bacteriophage λ, the Cre/loxP system from bacteriophage P1 and the FLP/FRT system from the Saccharomyces cerevisiae 2 mu circle plasmid. Bebee et al. (U.S. Pat. No. 5,434,066) discloses the use of site-specific recombinases such as Cre for DNA containing two loxP sites, used for in vivo recombination between the sites.

The recombinase specific site of the retroviral vector can be a site that is recognized by the Cre recombinase of bacteriophage P1, the FLP recombinase of Saccharomyces cerevisiae, the R recombinase of Zygosaccharomyces rouxii pSR1, the A recombinase of Kluyveromyces drosophilarium pKD1, the A recombinase of Kluyveromyces waltii pKW1, the integrase λInt, the recombinase of the GIN recombination system of the Mu phage, the bacterial β recombinase or a variant thereof. As mentioned above, the recombinase can be the Cre recombinase of bacteriophage P1 or its natural or synthetic variants. Cre is available commercially (Novagen, San Diego, Calif., USA, Catalog No. 69247). Recombination mediated by Cre is freely reversible. Cre works in simple buffers with either magnesium or spermidine as a cofactor, as is well known in the art. The DNA substrates can be either linear or supercoiled. A number of mutant loxP sites have been described. Such sites specific for said Cre recombinase can be chosen from the group composed of the sequences Lox P1, Lox 66, Lox 71, Lox 511, Lox 512, Lox 514, Lox B, Lox L, Lox R and mutated sequences of a Lox P1 site. The lox P sites can be heterotypic or homotypic. These sites allow Cre-mediated excision or replacement of the nucleic acid sequences in the vector with other sequences. For example, once the vector has integrated into the genome of the cell, one of skill in the art can contact the cell with Cre and remove the vector sequences to determine if cellular traits or phenotypes observed upon insertion of the vector were caused by loss of the gene occupied by the gene trap, thus providing a reversible gene trap. Similarly, FRT sites can be utilized such that a FLP recombinase can be utilized to excise the vector from the genome.

The vectors described herein contain appropriate packaging signals and can be prepared as virus particles containing the vectors packaged therein by using known packaging cell strains, for example, PG13 (ATCC CRL-10686), PG13/LNc8 (ATCC CRL-10685), PA317 (ATCC CRL-9078), cell strains described in U.S. Pat. No. 5,278,056, GP+envAm-12 (ATCC CRL-9641) and the like.

As set forth above, the vectors of the present invention comprise a nucleic acid sequence encoding a reporter protein. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), enhanced green fluorescent protein (EGFP), green reef coral fluorescent protein (G-RCFP), cyan fluorescent protein (CFP), red fluorescent protein (RFP or dsRed2), yellow fluorescent protein (YFP) and the like.

The vectors described herein also comprise an IRES site. The term “internal ribosome entry site” (IRES) defines a sequence motif which promotes attachment of ribosomes to that motif on internal mRNA sequences. Furthermore, all factors needed to efficiently start translation at the AUG-start-codon following said IRES attach to this sequence motif Consequently, an mRNA containing a sequence motif of a translation control element, e.g. IRES, results in two translational products, one initiating from the 5′ end of the mRNA and the other by an internal translation mechanism mediated by IRES. Accordingly, the insertion of a translational control element, such as IRES, operably linked to an ORF into a retroviral genome allows the translation of this additional ORF from a viral RNA transcript. Such RNA transcripts with the capacity to allow translation of two or more ORF are designated bi- or polycistronic RNA transcripts, respectively. IRES sequences are known in the art and include those from encephalomycarditis virus (EMCV) [Ghattas, I. R. et al., Mol. Cell. Biol., 11:5848-5849 (1991)]; BiP protein [Macejak and Sarnow, Nature, 353:91 (1991)]; the Antennapedia gene of Drosophila (exons d and e) [Oh et al., Genes & Development, 6:1643-1653 (1992)]; those in polio virus [Pelletier and Sonenberg, Nature, 334:320-325 (1988); see also Mountford and Smith, TIG, 11: 179-184 (1985)].

Further provided by the present invention is a method of selecting cells with homozygous mutations in their genomes comprising: a) contacting cells with a vector of the present invention, for example, a vector comprising a nucleotide sequence between a 5′ LTR and a 3′ LTR, wherein said nucleotide sequence comprises 1) an intron containing nucleic acid encoding a first selective marker operably linked to a promoter, 2) site specific recombinase sites, and 3) a 3′ exon comprising a nucleic acid encoding the 3′ segment of a second selective marker, an internal ribosome entry site (IRES), a nucleic acid encoding a reporter protein and a polyadenylation site; b) selecting cells with mutations induced by insertion of the vector into a cellular gene; c) exposing the cells to conditions that select for cells homozygous for vector-induced mutations; c) selecting cells that survive under the conditions of step c). The selection of cells with mutations induced by insertion of the vector into a cellular gene can be accomplished via routine selection methods such as drug resistance, for example, antibiotic resistance, in order to select those cells that have the vector inserted into a cellular gene and thus express a selectable marker. These cells can then be further analyzed for the presence of homozygosity.

The condition(s) that select for cells homozygous for vector-induced mutations can be increased drug resistance, for example, increased antibiotic concentration. For example, if the selectable marker is neomycin, one of skill in the art can select a concentration of G418 that allows selection of cells with a homozygous mutation. This concentration can be about 0.5 mg/ml, 0.6 mg/ml, 0.7 mg/ml, 0.8 mg/ml, 0.9 mg/ml, 1.0 mg/ml, 1.5 mg/ml, 2.0 mg/ml, 2.5 mg/ml, 3.0 mg/ml, 3.5 mg/ml, 4.0 mg/ml or any concentration in between. One of skill in the art can determine what selection condition are necessary for selection of cells that are homozygous for vector-induced mutations. The methods of the present invention are not limited to the use of the neomycin/G418 combination for selection of homozygous mutations. Any selectable marker, for example, other antiobiotic resistance genes, can be utilized in combination with an agent that allows selection of homozygous mutations. The cells that survive the condition(s) can be selected by one of skill in the art as cells that contain a homozygous mutation. Any cell from any organism can be mutated utilizing the methods of the present invention. The cell can be prokaryotic or eukaryotic, such as a cell from an insect, fish, crustacean, mammal, bird, reptile, yeast, or a bacterium such as E. coli. Exemplary cells include, but are not limited to, somatic cells, hematopoeitic cells, dividing cells, nondividing cells, embryonic stem cells, embryonic germ line cells, pluripotent stem cells and totipotent stem cells. The cell can be in vitro, in vivo or ex vivo.

Also provided by the present invention is a method of producing cells with increased frequency of homozygous mutations in their genomes comprising: a) contacting cells with a vector of the present invention; b) exposing the cells to a carcinogen; c) exposing the cells to conditions that select for cells homozygous for vector-induced mutations; and d) selecting cells that survive under the selective condition of step c). The above described method can optionally include a step of selecting cells with mutations induced by insertion of the vector into a cellular gene prior to exposing the cells to a carcinogen.

The present invention also provides a method of identifying an agent that increases the frequency of homozygous mutations in cells comprising: a) contacting cells comprising a vector of the present invention, wherein the vector is integrated into the genome of the cells, with the agent; b) exposing the cells to conditions that select for cells homozygous for vector-induced mutations; c) selecting cells that survive under the selective condition of step b); and d) determining the frequency of homozygous mutations, wherein if the frequency of homozygous mutations in cells contacted with the agent is greater than in cells not contacted with the agent, then the agent is an agent that increases the frequency of homozygous mutations in cells. This method can be utilized to identify a carcinogen or any other agent that increases the frequency of a homozygous mutation in cells. For comparison purposes, and in order to assess the effects of an agent, in the methods of the present invention, cells can be contacted with an agent in appropriate media or contacted with media alone. The cells contacted with media alone can be utilized as control cells. The agent can be, but is not limited to one or more of a drug, a chemical, a hormone, a small molecule, an antibody, a cDNA encoding a protein, an antisense molecule, an siRNA, a peptides or a protein. Two or more agents can also be used in combination; for example, a carcinogen known to increase the frequency of homozygous mutation can be used together with an siRNA that targets genes that could further enhance or suppress the frequency of homozygous mutation.

Also provided by the present invention is a method of identifying a compound that decreases the ability of an agent to enhance the frequency of producing homozygous mutations in cells comprising: a) contacting cells comprising a vector of the present invention, wherein the vector is integrated into the genome of the cells, with the compound and an agent that enhances the frequency of homozygous mutations in cells; b) exposing the cells to conditions that select for cells homozygous for vector-induced mutations; c) selecting cells that survive under the selective condition of step b); and d) determining the frequency of homozygous mutations, wherein if the frequency of homozygous mutations in cells contacted with the compound and the agent that increases the frequency of homozygous mutations is less than in cells contacted only with the agent that increases the frequency of homozygous mutations in cells, the compound is a compound that decreases the ability of an agent to increase or enhance the frequency of homozygous mutations in cells. The agent that increases the frequency of homozygous mutations can be a carcinogen. The compound that decreases the ability of an agent to enhance, or increase the frequency of producing homozygous mutant cells can be a drug used to prevent cancer or reduce damage to the genome associated with carcinogen exposure. This compound can be, but is not limited to a drug, a chemical, a hormone, a small molecule, an antibody, a cDNA encoding a protein, an antisense molecule, an siRNA, a peptides or a protein. A decrease in the ability of an agent to enhance the frequency of producing homozygous mutations does not have to be complete as this can range from a slight decrease to complete inhibition of the ability to increase the frequency of producing homozygous mutations. For example, the decrease can be about a 10%, 20%, 30%, 40%, 50%, 60%, 70%, 80%, 90% or 100% decrease.

Also provided by the present invention is a method of identifying cells that are homozygous for a mutation comprising: a) contacting cells with a vector of the present invention; b) exposing the cells to conditions that select for cells homozygous for vector-induced mutations; c) selecting cells that survive under the selective condition of step b); and d) isolating from the surviving cells a cellular gene within which the marker gene is inserted, thereby identifying cells that are homozygous for a mutation.

Further provided by the present invention is a method of identifying a gene, that when mutated, is associated with a recessive genetic trait and nonessential for cellular survival comprising: a) contacting cells with a vector of the present invention; b) exposing the cells to conditions that select for the genetic trait; c) selecting cells that survive and exhibit the genetic trait when gene function is decreased; and d) identifying the cellular gene disrupted by the vector.

This method allows the identification of genes that are associated with a recessive genetic trait when both alleles of the gene are mutated. A decrease in gene function can be, but is not limited to, a decrease in transcription of the gene, a decrease in translation, a decrease in expression, or a decrease in the activity of the gene product of the gene. The conditions that select for a genetic trait or phenotype can be determined by one of skill in the art depending on the genetic trait being analyzed. For example, one of skill in the art can expose the cells to a pathogenic organism in order to identify cells that survive. Cells that survive can be selected and the cellular gene disrupted in these cells can be identified as a gene that is involved in resistance to a pathogenic organism. In another example, the cells can be exposed to a toxin. Cells that survive exposure to the toxin comprise a gene that is disrupted by a vector of the present invention. This gene can be identified, thus identifying a gene that is involved in resistance to a toxin. Alternatively, if one of skill in the art is looking at the expression or function of a particular protein, for example, an enzyme, a cell surface protein, a receptor etc., cells can be assayed for the expression or function of the protein. Those cells with reduced expression or function of the particular protein can be selected and the gene disrupted by the vector of the present invention can be identified as a gene that is involved in the expression or function of that protein and thus associated with a phenotype that results from decreased gene expression or function. In yet another example, one of skill in the art can obtain or engineer cells that express a reporter protein when a particular pathway is active, for example, and not to be limiting, an enzymatic pathway, a metabolic pathway, a signal transduction pathway, or a pathway involved in pathogenesis. These cells can be contacted with a vector of the present invention. One of skill in the art can then determine if the vector has inserted itself into a gene that is involved in this pathway by monitoring reporter protein expression. If reporter protein expression changes, the disrupted gene can be identified as a gene that is involved in this pathway. For example, protein expression can increase or decrease.

In the methods of the present invention, the cells displaying the desired phenotype are selected for and depending upon the phenotype, the selection can be by a high throughput automated screening. For example, beads to select cells displaying a particular cell surface protein, such as a receptor. FACS analysis can also be used to identify the change in expression of particular receptors. As utilized throughout, a decrease in gene function or expression does not have to be complete as this can range from a slight decrease to complete inhibition of gene function or expression as compared to cells that do not have an insertion in a gene, that when mutated, is responsible for a recessive genetic trait. This decrease can be, for example, about 5%, 10%, 20%, 30%, 40%, 50%, 60%, 70%, 80%, 90% or 100%. An increase in gene function or expression can be about 5%, 10%, 20%, 30%, 40%, 50%, 60%, 70%, 80%, 90%, 100%, 200%, 300%, 400%, 500% or greater.

The method can optionally include the step of exposing the cells to conditions that select for cells homozygous for vector-induced mutations, such as, for example, increased antibiotic resistance. The method can optionally include the step of contacting the cells with an agent that increases the frequency of homozygous mutations in the cellular genome.

Therefore, also provided by the present invention is a method of identifying a gene responsible for a recessive genetic trait and nonessential for cellular survival comprising: a) contacting cells with a vector of the present invention; b) contacting the cells with an agent that increases the frequency of homozygous mutations in the cellular genome; c) selecting cells that survive and exhibit the genetic trait when gene function is decreased; and d) identifying the cellular gene disrupted by the vector. This method can optionally include selecting cells that survive under conditions that select cells with homozygous mutations prior to selection of cells that survive and exhibit the genetic trait when gene function is decreased. In the methods set forth herein, the recessive genetic trait can be any recessive genetic trait, including, but not limited to, cellular resistance to infection by a pathogenic organism, a trait involving the expression of a cell surface protein, a trait associated with signal transduction, a trait associated with the activity of an enzyme, a trait associated with a metabolic pathway, cellular resistance to a toxin, loss of cell growth control and loss of drug resistance, for example, resistance to cancer therapy drugs. As utilized herein, infection is not limited to entry of a pathogenic virus, but refers to all phases of pathogenic life cycles, For example, resistance to viral infection can involve viral attachment to cellular receptors, viral infection, viral entry, internalization, disassembly of the virus, viral replication, genomic integration of viral sequences, translation of mRNA, proteolytic cleavage of viral proteins or cellular proteins, assembly of viral particles, cell lysis and egress of virus from the cells

In the methods of the present invention, an agent that increases the frequency of homozygous mutations can be a chemical agent. These chemical agents include, but are not limited to alkylating agents, carcinogens and DNA damaging agents. Examples include, but not limited to, ethyl nitrosourea (ENU), 7,12-dimethly-1,2 benz[a]anthracene (DMBA), methyl-nitrosurea (MNU), hydroxyurea, doxoburubicin, diepoxybutane, cisplatin or mitomycin C. Radiation such as, ultraviolet irradiation or ionizing radiation, can also be utilized to increase the frequency of homozygous mutations.

Further provided by the present invention is a method of identifying a gene necessary for infection and nonessential for cellular survival comprising: a) contacting cells with a vector of the present invention; b) contacting the cells with a pathogen c) selecting cells that survive and exhibit resistance to infection when gene function is decreased; and e) identifying the cellular gene disrupted by the vector. This method can optionally include contacting the cells with an agent that increases the frequency of homozygous mutations in the cellular genome prior to, simultaneously with or after contacting the cells with a pathogen.

Therefore, the present invention provides a method of identifying a gene necessary for infection and nonessential for cellular survival comprising: a) contacting cells with a vector of the present invention; b) contacting the cells with an agent that increases the frequency of homozygous mutations in the cellular genome; c) contacting the cells with a pathogen d) selecting cells that survive and exhibit resistance to infection in the absence of gene function; and e) identifying the cellular gene disrupted by the vector.

This method can optionally include selecting cells that survive under conditions that select cells with homozygous mutations prior to selection of cells that survive and exhibit the resistance to viral infection. The present invention also provides the isolated nucleic acid of a gene identified via any of the methods of the present invention and an in vitro, in vivo or ex vivo cell comprising this isolated nucleic acid.

The pathogen can be a virus, a bacterium or a parasite. Examples of viral infections include but are not limited to, infections caused by all RNA viruses (including negative stranded RNA viruses, positive stranded RNA viruses, double stranded RNA viruses and retroviruses) and DNA viruses. Examples of viruses include, but are not limited to, HIV (including HIV-1 and HIV-2), parvovirus, papillomaviruses, measles, filovirus (for example, Ebola, Marburg), SARS (severe acute respiratory syndrome) virus, hantaviruses, influenza viruses (e.g., influenza A, B and C viruses), Dengue fever, hepatitis viruses A to G, caliciviruses, astroviruses, rotaviruses, reovirus, coronaviruses, (for example, human respiratory coronavirus and SARS coronavirus (SARS-CoV), picornaviruses, (for example, human rhinovirus and enterovirus), Ebola virus, human herpesvirus (such as, HSV-1-9, including zoster, Epstein-Barr, and human cytomegalovirus), foot and mouth disease virus, human adenovirus, adeno-associated virus, respiratory syncytial virus (RSV), smallpox virus (variola), cowpox, monkey pox, vaccinia, polio, viral meningitis and hantaviruses.

For animals, viruses include, but are not limited to, the animal counterpart to any above listed human virus, avian influenza (for example, strains H5N1, H5N2, H7N1, H7N7 and H9N2), and animal retroviruses, such as simian immunodeficiency virus, avian immunodeficiency virus, pseudocowpox, bovine immunodeficiency virus, feline immunodeficiency virus, equine infectious anemia virus, caprine arthritis encephalitis virus and visna virus.

Examples of bacteria include, but are not limited to, the following: Listeria (spp.), Mycobacterium tuberculosis, Rickettsia (all types), Ehrlichia, Chylamida. Further examples of bacteria that can be targeted by the present methods include M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Examples of parasites include, but are not limited to, the following: Cryptosporidium, Plasmodium (all species), American trypanosomes (T. cruzi). Furthermore, examples of protozoan and fungal species contemplated within the present methods include, but are not limited to, Plasmodium falciparum, other Plasmodium species, Toxoplasma gondii, Pneumocystis carinii, Trypanosoma cruzi, other trypanosomal species, Leishmania donovani, other Leishmania species, Theileria annulata, other Theileria species, Eimeria tenella, other Eimeria species, Histoplasma capsulatum, Cryptococcus neoformans, Blastomyces dermatitidis, Coccidioides immitis, Paracoccidioides brasiliensis, Penicillium marneffei, and Candida species.

Also provided by the present invention is a method of identifying a gene that is associated with a phenotype when homozygously mutated comprising: a) generating a mutant non-human animal comprising a homozygous mutation in a gene identified via the methods of the present invention; and b) determining a phenotype of the animal, thus identifying a gene that is associated with a phenotype.

The non-human animal can be, of any species, including, but not limited to, mice, chickens, rats, rabbits, guinea pigs, pigs, goats, sheep, teleosts (for example, zebrafish) and non-human primates, e.g., baboons, monkeys, and chimpanzees.

The present invention also provides a non-human transgenic mammal comprising a functional deletion of a gene identified via any of the methods of the present invention as necessary for infection, wherein the mammal has decreased susceptibility to infection by a pathogen, such as a virus, a bacterium, a fungus or a parasite. Exemplary transgenic non-human mammals include, but are not limited to, ferrets, fish, guinea pigs, chinchilla, mice, monkeys, rabbits, rats, chickens, cows, and pigs. Such knock-out animals are useful for reducing the transmission of viruses from animals to humans. In the transgenic animals of the present invention one or both alleles of a gene can be knocked out.

By “decreased susceptibility” is meant that the animal is less susceptible to infection or experiences decreased infection by a pathogen as compared to an animal that does not have one or both alleles of a gene necessary for infection knocked out or functionally deleted. The animal does not have to be completely resistant to the pathogen. For example, the animal can be 10%, 20%, 30%, 40%, 50%, 60%, 70%, 80%, 90%, 100% or any percentage in between less susceptible to infection by a pathogen as compared to an animal that does not have a functional deletion of the gene. Furthermore, decreasing infection or decreasing susceptibility to infection includes decreasing entry, replication, pathogenesis, insertion, lysis, or other steps in the replication strategy of a virus or other pathogen into a cell or subject, or combinations thereof.

Therefore, the present invention provides a non-human transgenic mammal comprising a functional deletion of a gene necessary for infection, wherein the mammal has decreased susceptibility to infection by a pathogen, such as a virus, a bacterium, a parasite or a fungus. A functional deletion is a mutation, partial or complete deletion, insertion, or other variation made to a gene sequence that inhibits production of the gene product or renders a gene product that is not completely functional or non-functional. Functional deletions can be made by insertional mutagenesis (for example via insertion of a transposon or insertional vector), by site directed mutagenesis, via chemical mutagenesis, via radiation or any other method now known or developed in the future that results in a transgenic animal with a functional deletion of a gene necessary for infection.

Alternatively, a nucleic acid sequence such as siRNA, a morpholino or another agent that interferes with mRNA expression can be delivered. The expression of the sequence used to knock-out or functionally delete the desired gene can be regulated by an appropriate promoter sequence. For example, constitutive promoters can be used to ensure that the functionally deleted gene is not expressed by the animal. In contrast, an inducible promoter can be used to control when the transgenic animal does or does not express the gene of interest. Exemplary inducible promoters include tissue-specific promoters and promoters responsive or unresponsive to a particular stimulus (such as light, oxygen, chemical concentration, such as a tetracycline inducible promoter).

The transgenic animals of the present invention can be examined during exposure to various pathogens. Comparison data can provide insight into the life cycles of pathogens. Moreover, knock-out animals (such as birds or pigs) that are otherwise susceptible to an infection (for example influenza) can be made to resist infection, conferred by disruption of the gene. If disruption of the gene in the transgenic animal results in an increased resistance to infection, these transgenic animals can be bred to establish flocks or herds that are less susceptible to infection.

Transgenic animals, including methods of making and using transgenic animals, are described in various patents and publications, such as WO 01/43540; WO 02/19811; U.S. Pub. Nos: 2001-0044937 and 2002-0066117; and U.S. Pat. Nos. 5,859,308; 6,281,408; and 6,376,743; and the references cited therein.

The transgenic animals of this invention also include conditional gene knockdown animals produced, for example, by utilizing the SIRIUS-Cre system that combines siRNA for specific gene-knockdown, Cre-loxP for tissue-specific expression and tetracycline-on for inducible expression. These animals can be generated by mating two parental lines that contain a specific siRNA of interest gene and tissue-specific recombinase under tetracycline control. See Chang et al. “Using siRNA Technique to Generate Transgenic Animals with Spatiotemporal and Conditional Gene Knockdown.” American Journal of Pathology 165: 1535-1541 (2004) which is hereby incorporated in its entirety by this reference regarding production of conditional gene knockdown animals.

The present invention also provides cells including an altered or disrupted gene, wherein the gene is identified via the methods of the present invention, that are resistant to infection by a pathogen. These cells can be in vitro, ex vivo or in vivo cells and can have one or both alleles altered. These cells can also be obtained from the transgenic animals of the present invention. Such cells therefore include cells having decreased susceptibility to HIV infection, Ebola infection, avian flu, influenza A or any of the other pathogens described herein, including bacteria, parasites and fungi.

The following examples are put forth so as to provide those of ordinary skill in the art with a complete disclosure and description of how the nucleic acids, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

### Example I

The present invention provides methods for biallelic mutagenesis in mammalian cells. Novel poly(A) gene trap vectors, which contain features to facilitate the identification of disrupted genes and for post-entrapment genome engineering, were used to generate a library of 980 mutant ES cells. The entrapment mutations generally disrupted gene expression and were readily transmitted through the germline, establishing the library as a resource for constructing mutant mice. Cells homozygous for most entrapment loci could be isolated by selecting for enhanced expression of a inserted neomycin resistance gene that resulted from losses of heterozygosity (LOH). The frequencies of LOH measured at 37 sites in the genome ranged from 1.3×10⁻⁵ to 1.2×10⁻⁴ per cell and increased with increasing distance from the centromere, implicating mitotic recombination in the process. The ease and efficiency of obtaining homozygous entrapment mutations (i) facilitates genetic studies of gene function in cultured cells, (i) permits genome-wide studies of recombination events that result in LOH and mediate a type of chromosomal instability important in carcinogenesis, and (iii) provides new strategies for phenotype-driven mutagenesis screens in mammalian cells.

### Entrapment Vectors

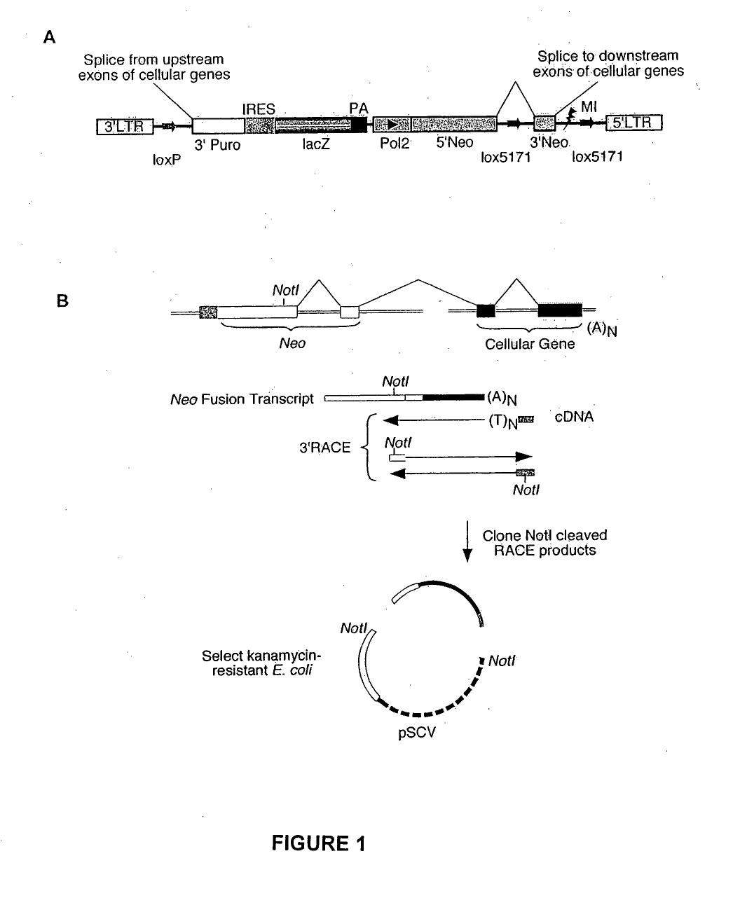

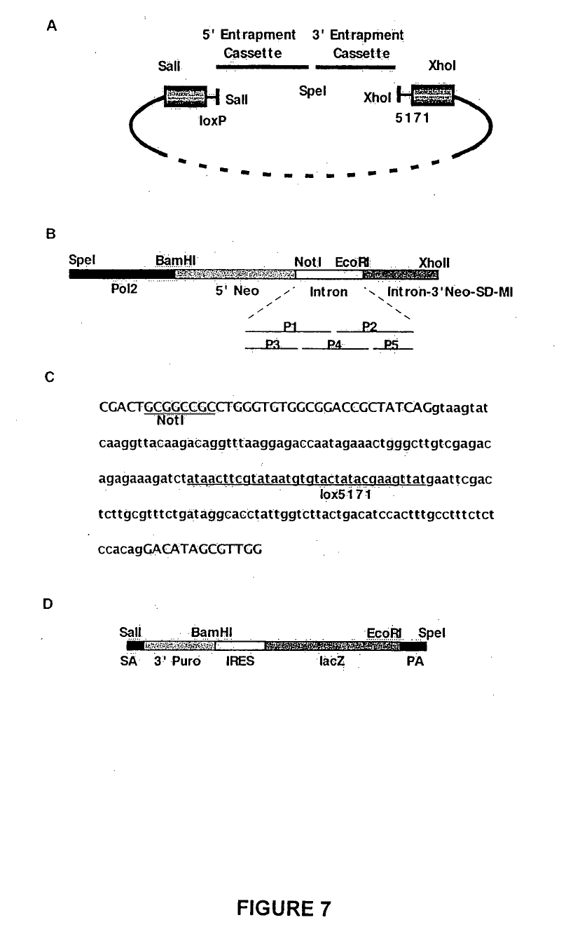

GTRx.x entrapment vectors (FIGS. 1a and 7a) function as 3′ gene (or PolyA) traps (18-20,27). The GTR vectors were constructed as shown in FIG. 7a. The plasmid/vector backbone for GTR gene trap retroviruses, which includes both LTRs and flanking wild type and 5171 loxP sites was derived by cleaving LNPAT1 (see Reference 23 (Osipovich et al.) for construction of LNPAT1 vector) with SalI and XhoI.

The 3′ entrapment cassette (FIG. 7b) was constructed from three elements. (1) A fragment containing the Pol2 promoter and 5′ end of Neo gene was amplified from LNPAT1 using the SpeI-Pol2-Neo and Pol2-Neo-NotI primers. This introduced a synthetic NotI site introduced at position 776 of the Neo sequence (Genbank accession V00618). (2) 5 oligonucleotides (P1-P5) were annealed to produce a sequence flanked by NotI and EcoR1 sites that contains the 5′ end of an intron inserted after nucleotide 807 of the V00618 sequence (FIG. 7c). (The promoter region and 5′ end of the kanamycin resistance gene (Neo) were amplified from pCR4-TOPO (Invitrogen; Genbank accession AX806464) using the SacIIPro and NeoNotI primers, and the PCR product was cloned between the SacII and NotI sites of pBluescript II KS(−) (Stratagene). The NeoNotI primer introduces two nucleotide substitutions in the Neo sequence, creating a NotI site without altering the Neo protein coding sequence. Specifically, the T at position 2296 and the G at position 2301 in the AX806464 sequence were both converted to C (FIG. 7c).

(SEQ ID NO: 20)

SacIIPro:
AGAGAGAAGCTTTCAGCGGCCGCAGTCGATGAATCCAGAAAAGCG

(SEQ ID NO: 21)

NeoNotI:
AGAGAGCCGCGGATGGCGATAGCTAGACTGGGCGG)

(3) A fragment from the 3′ end of the Neo entrapment cassette from LNPAT1 was amplified using primers EcoRI-Neo3′-SD-MI and Pol2-Neo-SD-MI-XhoI (and in a nested reaction primers EcoRI-Neo(nest) and Pol2-Neo-SD-MI-XhoI). The EcoRI-Neo3′-SD-MI primer provides the 3′ end of the inserted intron. The resulting sequence is identical to the 3′ entrapment cassette of LNPAT1 except for the insertion of a NotI site and intron in the Neo coding sequence as shown in FIG. 7c. For GTR2.x vectors the SpeI-BamHI fragment containing the Pol2 promoter was replaced with the PGK promoter.

The 5′ entrapment cassette (in GTR1.0-GTR1.3 and GTR2.0-2.3) was derived from the 5′ entrapment cassette from LNPAT1 except the 3′ end of the puromycin resistance gene (Pac, nucleotides 443-853 of the Genbank M25346 sequence) was amplified by primers (SalI-SA-Puro3′ and SA-puro3′-BamHI) that contained a splice acceptor sequence and ligated at the BamHI site to the IRES-lacZ-poly(A) sequence amplified using BamHI-IRES-LacZ-PA and IRES-LacZ-PA-SpeI (FIG. 7d). Alternatively the BamHI-EcoRI fragment containing the IRES-lacZ sequence was replaced by an EGFP reporter (GTR1.4-GTR1.7 and GTR 2.4-GTR2.7).

SpeI-Pol2-Neo:

(SEQ ID NO: 22)

AGAGAGACTAGTGGGCTGAACATCGAGCGCCAGGGC
Pol2-Neo-NotI:

(SEQ ID NO: 23)

CGCCACACCCAGGCGGCCGCAGTCGATGAATCCAGAAAAGCGG
EcoRI-Neo3′-SD-MI:

(SEQ ID NO: 24)

AGAGGAATTCGACTCTTGCGTTTCTGATAGGCACCTATTGGTCTTACTGA
CATCCACTTTGCCTTTCTCTCCACAGGACATAGCGTTGGCTACCCGTGAT
EcoRI-Neo(nest):

(SEQ ID NO: 25)

AGAGGAATTCGACTCTTGCGTTTCTG
Pol2-Neo-SD-MI-XhoI:

(SEQ ID NO: 26)

CTCTCTCTCGAGGCTTAAATAAATAAATAAATAAATAT
SalI-SA-Puro3′:

(SEQ ID NO: 27)

AGAGAGGTCGACGACTCTTGCGTTTCTGATAGGCA
SA-puro3′-BamHI:

(SEQ ID NO: 28)

CTCTCTGGATCCTCAGGCACCGGGCTTGCGGGTCA
BamHI-IRES-LacZ-PA:

(SEQ ID NO: 29)

AGAGAGGGATCCGCCCCTCTCCCTCCCCCCCCCCTA
IRES-LacZ-PA-SpeI:

(SEQ ID NO: 30)

TGTCCAAACTCATCAATGTATCTTACTAGTAGAGAG

The virus inserts a Neo gene throughout the genome, and selection for G418 resistance generates clones in which Neo sequences splice to 3′ distal exons of cellular genes. The Neo gene was expressed either from the Pol2 (GTR1.x) or PGK (GTR2.x) promoters; hence, like other poly(A) traps (20,23), the vectors can target genes that are not expressed in ES cells. Expression of the occupied cellular genes is disrupted by a 3′ exon consisting of sequences from the 3′ end of a puromycin resistance gene, an internal ribosome entry site and a reporter protein [either a nuclear β-galactosidase (lacZ; GTR1.0-GTR1.3) or enhanced green fluorescent protein (EGFP; GTR1.4-GTR1.7)]. Wild-type and mutant [lox5171, (28)] loxP sites allow provirus inserts to be engineered by recombinase-mediated cassette exchange (RMCE) (22,23). GTR1.0 and GTR1.3 contain an additional loxP5171 site located in a synthetic intron inserted into the Neo gene (FIG. 7c). The 3′ Puro segment provides the 3′ end of a split puromycin resistance gene and, when used in combination with the 5′ end of the gene, is designed to select for Cre-mediated inter- and intra-chromosomal recombination events, as has been described using a split Hprt gene (25,26). The split Neo gene in GTR1.0 and GTR1.3 can also used for this purpose if the 3′ Neo exon is first deleted via recombination at the lox5171 sites.

The Neo gene was engineered by inserting an intron and a NotI cleavage site by site-directed mutagenesis; neither modification affected the protein coding sequence (FIG. 7c). These features allow 3′RACE products from spliced fusion transcripts to be cloned directly in E. coli (FIG. 1B). Briefly, fusion transcripts amplified by 3′RACE are cleaved with NotI and ligated to a plasmid (pSCV) containing Neo sequences upstream of the NotI site under the control of a strong bacterial promoter. Bacterial clones containing the desired RACE products are then selected on kanamycin plates. All steps in the process (expansion and cryopreservation of neomycin-resistant ES clones, RNA extraction, 3′RACE, and DNA sequencing) were performed in a 96-well format. Fusion transcripts were cloned from 70-80% of ES cells grown in single well without using nested PCR or highly competent E. coli, equivalent to the efficiency of cloning 3′ RACE products manually (23).

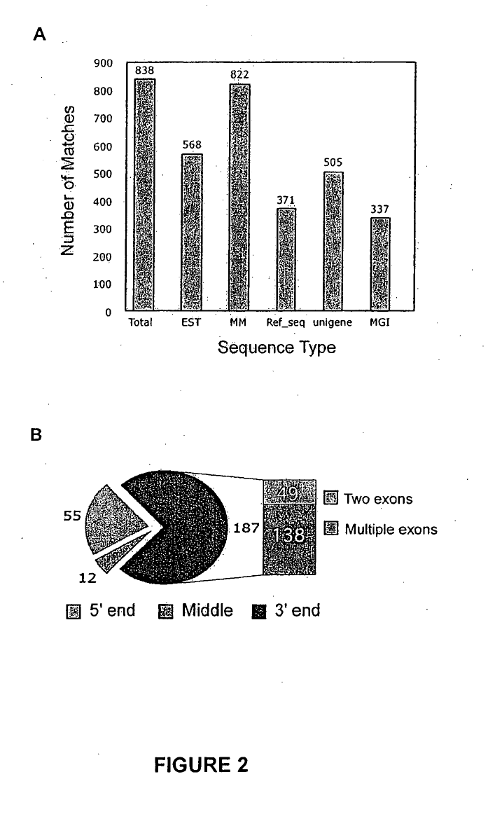

3′ RACE products from 980 ES cell clones were cloned, sequenced and compared against the mouse genome, EST and RefSeq databases using MultiBlaster, a relational database for performing BLAST searches on large numbers of DNA sequences (29). 58 sequences contained repetitive DNA and were not informative. Of the remaining 922 sequences 903, 645, 609, and 438 returned significant matches (nearly all matches had p values greater than 10⁻⁵⁰, and none were less than 10⁻²⁰) with sequences in the mouse genome, EST and RefSeq databases (FIG. 2). 539 matched sequences for which a unigene number has been designated and 349 matched MGI reference genes. Approximately 35% of the cloned 3′ RACE products matched mouse genomic sequences (MGSCv3) for which their was no annotation to suggest that the provirus had inserted into a previously characterized gene. Some of these inserts may reflect the presence of either new genes or additional exons that would extend the boundaries of adjacent transcription units. In addition, cryptic 3′ exons not normally associated with annotated genes may also be capable of supporting Neo gene expression, as suggested by intron-derived RACE products that are in the opposite transcriptional orientation to that of the occupied gene. These results are consistent with recent transcriptional maps suggesting that much of transcribed genome is not associated with annotated genes (30). For example, 56% of cytoplasmic polyadenylated RNAs do not contain annotated exon or intron sequences. As previously noted for other poly(A) traps (23,31) for inserts involving well-annotated genes, GTR vectors preferentially targeted the last intron and expressed fusion transcripts that spliced to a single downstream exon. However, the preference was less pronounced, as 26% of the inserts in well-characterized genes were in upstream introns (FIG. 2B).

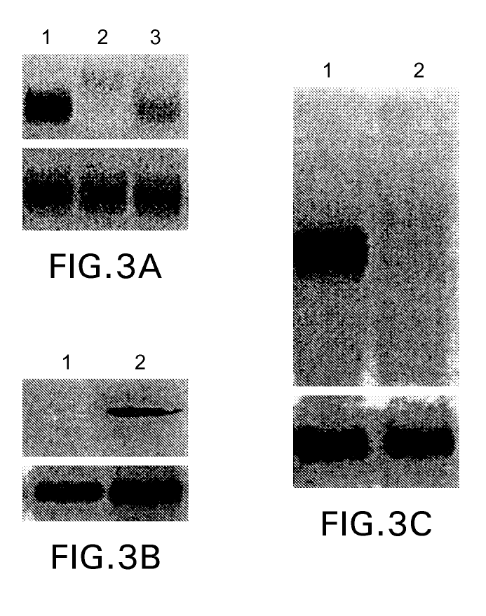

Clones from the entrapment library were highly germline competent as all 10 entrapment loci that have been tested to date were readily transmitted through the germline. The GTR vectors appeared to be effective mutagens as 3 of 9 inserts into annotated genes induced obvious phenotypes when bred to a homozygous state. Specifically, an insert into Hesx1 produced similar defects in eye development to those described for a targeted null mutation (32); the Dymeclin mutation caused defects in bone growth similar to defects observed in humans (33); and animals homozygous for the Pfdn1 mutation die within 5 weeks of age. In all cases examined (Cradd, Dymeclin and Pfdn1), entrapment mutagenesis significantly ablated expression of the occupied allele (FIG. 3). Thus, the entrapment library provides a resource from which mutations in genes of interest can be selected for transmission into the germline. The mutations have been contributed to the International Gene Trap Consortium [IGTC, (2)], and the 3′RACE sequences have been submitted into the GSS Genbank database (Accession numbers CZ169539 to CZ170518). Mutations in specific genes can be identified by searching either the IGTC or GSS databases, and the corresponding ES cell clones are available on request.

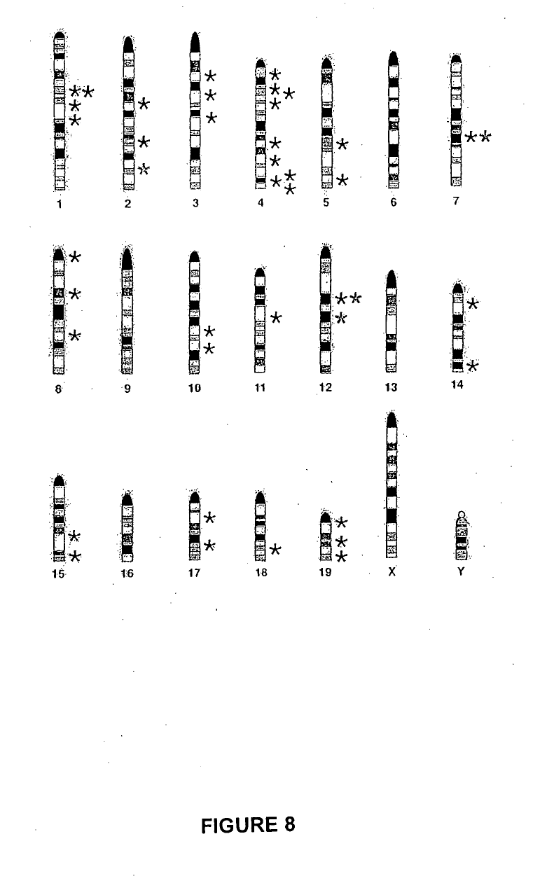

Whether homozygous mutants could be selected from the GTR entrapment library was tested. The influence of chromosome location on the frequencies of LOH was also examined. 37 clones with random entrapment mutations (FIG. 8 and Table 1) induced by GTR1.3 were placed in media containing 2.0 mg/ml G418 to select potential clones that had undergone spontaneous LOH. The frequencies of resistance to high G418 ranged from 1.3×10⁻⁵ to 1.2×10⁻⁴ similar to the frequencies reported for Neo genes inserted by homologous recombination (15,17) and for cellular loci at which LOH frequencies can be measured (11-13,34). Optimal levels of G418 used for selection were determined by pilot experiments, and 2.0 mg/ml provided the best combination of yield and specificity for cells mutagenized with GTR1.3. However, entrapment clones containing the GTR2.3 vector (in which Neo is expressed from the PGK promoter) displayed greater resistance to G418, and there was insufficient killing even at 3.0 mg/ml G418 to recover clones that had undergone LOH (Table 1). Levels of neomycin resistance correlate with levels of Neo gene expression (16); thus, use of the Pol2 promoter, which is four times weaker than the PGK promoter (23), can be an important variable with regard to the reliable selection of potential homozygous mutant entrapment clones. However, the present invention is not limited to the use of the Pol2 promoter and contemplates the use of other promoters to drive expression of one or more selective markers in the vectors of the present invention.

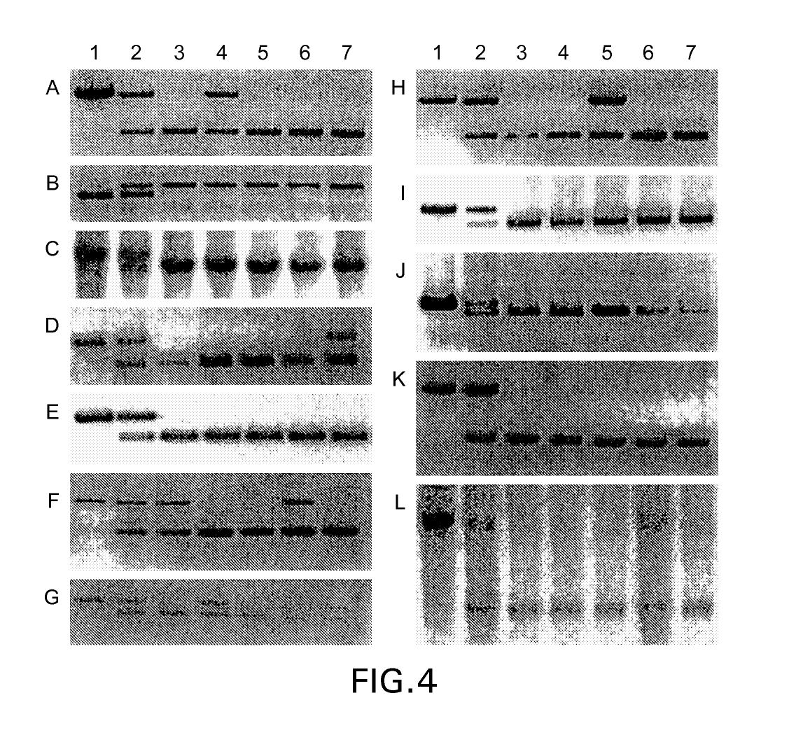

Genotypic analysis of 12 different mutants induced by GTR1.3 confirmed that a significant proportion (82% overall) of the colonies surviving in 2.0 mg/ml G418 had undergone LOH (FIG. 4). Cells homozygous for all 12 entrapment loci tested were recovered at frequencies ranging from 40-100% of the high G418-resistant colonies arising from each clone. The 12 entrapment clones were randomly selected based on the availability of flanking sequence probes and primers capable of distinguishing the wild-type and occupied alleles. Since entrapment clones generated by GTR1.3 produced colonies resistant to 2.0 mg/ml G418 at similar frequencies (Table 1), and a high proportion of the high G418-resistant colonies analyzed from each tested clone had undergone LOH, it was concluded that most mutations in the stem cell library can be converted to a homozygous state.

By contrast, only one GTR1.3 entrapment clone was encountered for which homozygous mutant ES cells could not be isolated. These experiments (Cao et al., unpublished) were performed as part of a separate study to characterize a mutation in Pfdn1 (prefoldin), a chaperone that assists in the folding of cytoskeletal proteins (36). Briefly, cells heterozygous for the Pfdn1 mutation formed colonies in 2.0 mg/ml G418 at frequencies (5.6×10⁻⁶) more than 10-fold lower than observed with other GTR1.3 entrapment clones and similar to the frequencies observed with ES cells heterozygous for a targeted mutation in Ssrp1, which encodes a chromatin remodeling protein essential for ES cell viability (35). Moreover, the colonies arising in high G418 were small, and the cells could not be propagated further. Therefore, prefoldin appears to be required for the clonal outgrowth of ES cells, accounting for the failure to isolate homozygous mutant cells.

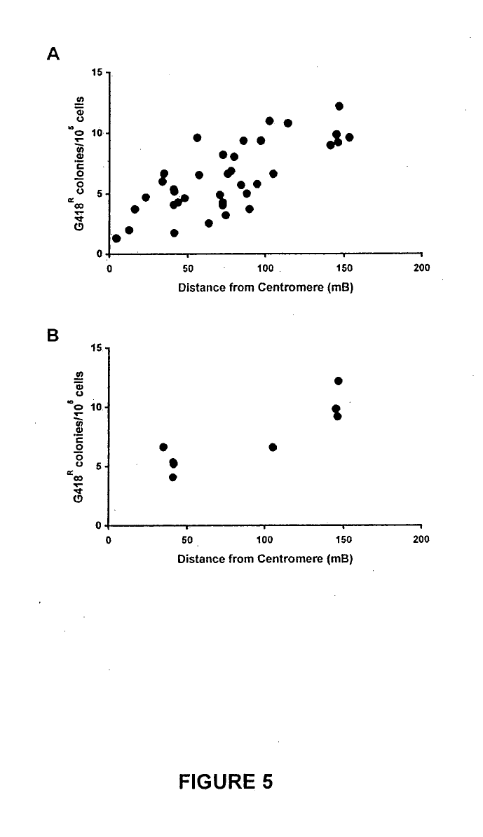

The frequency of colony formation in high G418 increased with increasing distance from the centromere (FIG. 5). The R² by linear regression analysis was 0.54 for all genes in aggregate (FIG. 5a) and 0.78 for the eight loci on chromosome 4 considered separately (FIG. 5b). The influence of chromosome position was observed despite many potential variables that could influence the induction or recovery of clones with LOH [e.g. levels of Neo expression, DNA sequence effects on recombination, or clonal variation in plating efficiencies ranging from 30 to 50%]. The chromosome position effect suggests that mitotic recombination plays a significant role in spontaneous LOH, as previously observed for Neo genes inserted by gene targeting in ES (17) and for the APRT gene in other cell types (12,13).

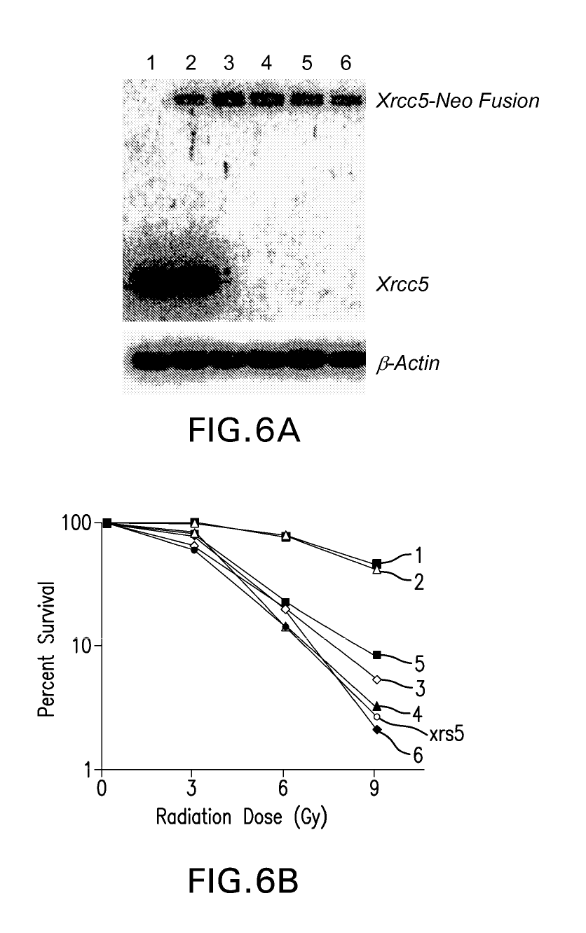

The ease and efficiency of obtaining homozygous entrapment mutations will enhance the utility of mutant ES cell libraries in several ways. First, cells deficient in any gene of interest (assuming the gene is expressed in ES cells and is not required for cell viability) can be readily obtained for biochemical and metabolic studies of gene function, without the time or expense of introducing the mutation into the germline. For example, LOH involving a mutation in the Xrcc5 gene (FIG. 4) resulted in a complete loss of Xrcc5 expression in the homozygous mutant cells (FIG. 6a). As expected the Xrcc5-deficient cells were also hypersensitive to γ-irradiation as compared to the parental or heterozygous mutant ES cells (FIG. 6b). Second, analysis of homozygous mutants is useful in assessing whether gene entrapment has induced a null mutation as illustrated by the Xrcc5 mutation (FIG. 6a). Third, the ability to select homozygous mutant cells will provide an early assessment of whether the disrupted genes or chromosome deletions engineered post entrapment are required for cell viability. Fourth, allelic imbalance is a common manifestation of chromosome instability in human cancers, which may harbor over 10,000 regions of LOH per cell (37). The source of this genome-wide LOH is unknown and unfortunately, frequencies of LOH at specific sites is typically measured at relatively few loci (e.g. Tk, Aprt, Hprt or cell surface antigens) where gene inactivation confers a selectable phenotype. Entrapment ES cell clones provide resources to study factors, such as carcinogens, localized elements in the genome or in genes required for genome maintenance (disrupted by mutation or RNA interference), that influence the frequencies of LOH at many sites throughout the genome.

Finally, losses of heterozygosity involving GTR poly(A) traps will assist phenotype-driven mutagenesis screens in mammalian cells (6,7,38). Mutagens incorporating Pol2Neo as an LOH selection cassette, should facilitate the recovery homozygous mutants by combining selection for high G418 resistance together with strategies that enhance the frequencies of LOH (7,38). Alternatively, since losses of heterozygosity involving inserted Neo resistance genes extend across large chromosome regions (17), stem cell clones containing inserted GTR1.3 vectors can be used to enhance the recovery of homozygous recessive mutants located on the same chromosome as the entrapment vector. This provides an alternative to the use of site-specific recombinases to induce mitotic recombination (9,10), eliminating the need to insert recombinase target sequences at allelic sites in the genome.

### Gene Entrapment

AC1 mouse embryonic stem cells were derived from an explanted 129svJ blastocyst cultured on feeder layers of irradiated mouse embryo fibroblasts and were cultured as described previously (39).

Construction of GTR poly(A) trap vectors is described herein. Retroviruses were prepared by transfecting GTR plasmids into Phoenix Eco cells by calcium phosphate coprecipitation. Virus production by individual clones was titered as Neo^{R} colony-forming units in NIH3T3 cells(23). Supernatants from producer lines with titers of 200 Neo^{R} CFU/mL were used to infect AC1 ES cells. 24 h post-infection, the cells were placed in selective media containing 250 μg/mL of G418 (Invitrogen) and cultured for 7 days during which the media was changed every day. Individual G418 resistant colonies were transferred to a single well of a 96 well plate. After 3-5 days the plates were passaged to three 96 well plates and grown for an additional 2-3 days. One plate was used to prepare RNA for 3′RACE and two plates were cryopreserved in liquid nitrogen.

### Identification of Genes Disrupted by Gene Entrapment

Disrupted genes were identified by sequencing cloned Neo fusion transcripts amplified by 3′-RACE. Total RNA was extracted using the RNeasy96 system (Qiagen Ltd, Dorking, England) according to the manufacturer's instructions. cDNA was synthesized using the superscript II reverse transcrtiptase (Invitrogen) in a 20-μL reaction containing 1 μg of total RNA and an NotI-adaptor-oligo-dT primer (5′-GACTAACCCGGCTCGAGCGGCCGCTTTTTTTTTTTTTTTTTT-3′) (SEQ ID NO: 31). The cDNA was then amplified by two rounds of PCR in a 50-μL reaction using the hotstart Taq polymerase kit (Qiagen). The PCR reactions contained 2 μL of the above cDNA product with a Neo-specific primer (5′-ATGGCCGCTTTTCTGGATTCATCG-3′) (SEQ ID NO: 32) and NotI adaptor primer (5′-GACTAACCCGGCTCGAGCGGCCGCT-3′) (SEQ ID NO: 33). All the reactions were performed in 96 well plates. PCR products were purified using QIAquick 96 cartridges (Qiagen) and digested with NotI for 1 hour and then purified again over QIAquick 96.

NotI digested 3′RACE products were ligated together with the selective cloning vector (PSCV, see Supporting Information) and transferred into chemically competent DH5α E. coli. The transformed cells were cultured for two hours in LB media and plated on LB agar plates containing 250 μg/ml kanamycin. Individual colonies were picked and grown in 96-well mL LB media overnight. Plasmids were prepared with QIAprep 96 Miniprep cartridges (Qiagen) and sequenced(23), using a Neo-specific primer: TCCCGATTCGCAGCGCATCGCC (SEQ ID NO: 34). When entrapment clones were grown on Neo-resistant feeder layer cells, 3′ RACE also generated small inserts, of 110 nt in size, generated by recombination between the fusion transcripts (which contain a NotI site) and Neo transcripts expressed by the feeder cells. To eliminate this background, plasmids were pre-screened to identify clones with larger RACE products.

### Isolation of Flanking Genomic DNA

Flanking genomic DNA sequences were cloned by inverse PCR (40) or by ligation-mediated PCR (41) modified by the addition of a C3 spacer (Integrated DNA Technologies) to the NlaIII minus adaptor to block the amplification of fragments via adaptor primers alone. Briefly, genomic DNA was (i) digested with NlaIII, ligated to a 1:1 mixture of NlaIII plus (5′-GTAATACGACTCACTATAGGGCTCCGCTTAAGGGACCATG-3′) (SEQ ID NO: 35) and minus (5′-Phos-GTCCCTTAAGCGGAG-C3-spacer (SEQ ID NO: 36)) strand adaptors, (ii) digested with PstI to prevent amplification of sequences from the 5′ LTR and (iii) subjected to two rounds (30 cycles each) of PCR using nested primers to the LTR and adaptor sequences.

| | |
|---|---|
| First round PCR, LTR: | |
| 5′-GCTAGCTTGCCAAACCTACAGGTGG-3′ | (SEQ ID NO: 37) |
| Adaptor: | |
| 5′-GTAATACGACTCACTATAGGGCTCCG-3′ | (SEQ ID NO: 38) |
| Second round PCR, LTR: | |
| 5′-CCAAACCTACAGGTGGGGTCTTTC-3′ | (SEQ ID NO: 39) |
| Adaptor: | |
| 5′-AGGGCTCCGCTTAAGGGAC-3′). | (SEQ ID NO: 40) |

### Selection and Analysis of LOH

Serially diluted cells were plated in triplicate onto 150 mm plates and allowed to attach overnight. Subsequently, unattached cells were removed and selection media containing either 0.0, 0.3, or 2.0 mg/ml G418 was added to each dish. After 12 days, the number of colonies surviving in each dish was counted, and the frequency of colony formation at 2.0 mg/ml G418 was determined by dividing the number of colonies obtained from 0.3 mg/ml G418 selection to that obtained from 2.0 mg/ml G418 selection.

The genotypes of clones surviving in 2.0 mg/ml G418 were determined by Southern blot and PCR analysis. For Southern blot analysis 5 μg of endonuclease cleaved DNA was fractionated on 0.9% (w/v) agarose gels and hybridized to probes genomic DNA sequences adjacent to the entrapment vector. For PCR analysis, 200 ng of genomic DNA was amplified using two primers complementary to genomic DNA located on either side of the site of provirus insertion and one primer specific for the entrapment vector.

### Analysis of Xrcc5 Entrapment Clones

Serially diluted cells were plated in triplicate onto 150 mm plates and allowed to attach overnight. Subsequently, cells were irradiated in culture medium at a dose rate of 3 Gy/min (200 kV, 4 mA, 0.78 mm A1). Colonies were counted at 12 days after irradiation, and the percent surviving was determined relative to numbers of colonies from untreated cells.

TABLE 1

| Clone | GSS | 2 mg/ml G418 | Chromosomal | Gene Disrupted |
|---|---|---|---|---|
| ID | Accession # | only | Location | and Gene ID or MGI |
| b3p3-d8 | CZ169573 | 2.53 × 10⁻⁵ | 1C2 | ND |
| b3p3-d4 | CZ169572 | 3.20 × 10⁻⁵ | 1C3 | LOC227288 (2448715) |
| b3p4-d12 | CZ169762 | 4.05 × 10⁻⁵ | 1C3 | (MGI: 104517) |
| b3p4-g3 | CZ169810 | 4.28 × 10⁻⁵ | 1C3 | Xrcc5 (MGI: 104517) |
| b3p4-g9 | CZ169804 | 4.91 × 10⁻⁵ | 2C2 | Grb14 (Mm.33806) |
| b3p4-e1 | CZ169763 | 9.59 × 10⁻⁵ | 2G3 | KIF3B (MGI: 107688) |
| *b5p5-c1 | CZ170037 | 1.18 × 10⁻¹ | 2H1 | Cdk5rap1 (MGI: 1914221) |
| b3p4-b12 | CZ169783 | 9.59 × 10⁻⁵ | 3A1 | ND |
| b3p4-b1 | CZ169784 | 3.73 × 10⁻⁵ | 3A2 | S12207 hypothetical protein |
| b3p4-a5 | CZ169854 | 1.74 × 10⁻⁵ | 3B | ND |
| b3p3-b6 | CZ169662 | 5.00 × 10⁻⁵ | 3F1 | ND |
| b3p4-a1 | CZ169780 | 6.67 × 10⁻⁵ | 4A4 | 1810030N24Rik (MGI: 1913541) |
| b3p3-a4 | CZ169660 | 5.20 × 10⁻⁵ | 4A5 | (Mm.96573) |
| b3p3-a9 | CZ169623 | 5.37 × 10⁻⁵ | 4A5 | (MGI: 3045357) |
| b2p1-b9 | CZ169682 | 4.07 × 10⁻⁵ | 4B1 | Spink4 gene (MGI: 1341848) |
| b3p4-b8 | CZ169787 | 6.60 × 10⁻⁵ | 4C6 | ND |
| *b5p9-a5 | CZ170167 | 1.43 × 10⁻¹ | 4D2.3 | Smpdl3b (MGI: 1916022) |
| b3p3-g10 | CZ169605 | 1.22 × 10⁻⁴ | 4E1 | ND |
| b3p4-f2 | CZ169770 | 9.20 × 10⁻⁵ | 4E1 | Mad212 (MGI: 1919140) |
| b3p3-d9 | CZ169663 | 9.84 × 10⁻⁵ | 4E2 | D4Cole1e gene |
| b3p3-h4 | CZ169643 | 3.71 × 10⁻⁵ | 5E2 | D430040L24Rik (MGI: 2444469) |
| b3p3-c12 | CZ169567 | 8.97 × 10⁻⁵ | 5G2 | D130017N08Rik (2443273) |
| b3p4-f6 | CZ169773 | 9.33 × 10⁻⁵ | 7E3 | 1600010M07Rik (MGI: 1917031) |
| *b5p9-d2 | CZ170185 | 1.58 × 10⁻¹ | 7E3 | 1600010M07Rik (MGI: 1917031) |
| b3p4-h11 | CZ169856 | 2.00 × 10⁻⁵ | 8A1.1 | 4933439N14Rik (Mm.160052) |
| b3p4-f10 | CZ169812 | 4.64 × 10⁻⁵ | 8A4 | ND |
| b3p4-c2 | CZ169794 | 9.34 × 10⁻⁵ | 8C3 | (Mm.24524) |
| b3p4-g8 | CZ169802 | 5.71 × 10⁻⁵ | 10C1 | Rfx4 (MGI: 1918387) |
| b3p3-g6 | CZ169601 | 5.78 × 10⁻⁵ | 10C2 | Cradd (MGI: 1336168) |
| b3p4-b2 | CZ169785 | 6.53 × 10⁻⁵ | 11B1.3 | 2010001A14Rik (MGI: 1923766) |
| *b5p9-h9 | CZ170207 | 1.23 × 10⁻¹ | 12C1 | Mipol1 (MGI: 1920740) |
| b2p1-a5 | CZ169683 | 6.62 × 10⁻⁵ | 12C3 | Galntl1 (MGI: 1917754) |
| *b5p6-h2 | CZ170405 | 7.28 × 10⁻² | 12C3 | Galntl1 (MGI: 1917754) |
| b3p3-c8 | CZ169557 | 4.72 × 10⁻⁵ | 14A3 | Hesx1 gene (MGI: 96071) |
| b3p4-d10 | CZ169761 | 1.08 × 10⁻⁴ | 14E5 | Phgdhl1 (MGI: 1916139) |
| b3p4-e3 | CZ169766 | 6.85 × 10⁻⁵ | 15D3 | ND |
| b3p4-c9 | CZ169841 | 1.10 × 10⁻⁴ | 15F2 | ND |
| b3p4-c1 | CZ169790 | 4.29 × 10⁻⁵ | 17B3 | LOC433110 (Mm.45676) |
| b3p4-c12 | CZ169852 | 8.20 × 10⁻⁵ | 17E2 | ND |
| b3p1-h1 | CZ169622 | 8.02 × 10⁻⁵ | 18E2 | 4933427L07Rik (MGI: 1918480) |
| b3p3-h8 | CZ169641 | 1.31 × 10⁻⁵ | 19A | RBM4 (MGI: 1100865) |
| b3p3-h2 | CZ170481 | 6.02 × 10⁻⁵ | 19C1 | AW210596(MGI: 2147716) |

*GTR2.3 vector
ND = no data

### Example II

Widespread losses of heterozygosity (LOH) in human cancer have been thought to result from chromosomal instability caused by mutations affecting DNA repair/genome maintenance. However, the origin of LOH in most tumors is unknown. The present study examined the ability of carcinogenic agents to induce losses of heterozygosity (LOH) at 53 sites throughout the genome of normal diploid mouse embryo-derived stem (ES) cells. Brief exposures to non-toxic levels of methyl-nitrosourea, diepoxybutane, mitomycin C, hydroxyurea, doxorubicin, and UV light stimulated LOH at all loci at frequencies ranging from 1-8×10⁻³ per cell (10 to 123 times higher than in untreated cells). These results suggest that LOH contributes significantly to the carcinogenicity of a variety of mutagens, and raises the possibility that genome-wide LOH observed in some human cancers may reflect prior exposure to genotoxic agents rather than a state of chromosomal instability during the carcinogenic process. Finally as a practical matter, chemically induced LOH is expected to enhance the recovery of homozygous recessive mutants from phenotype-based genetic screens in mammalian cells.

Cancer is thought to arise from the accumulation of somatic mutations in oncogenes and tumor suppressor genes that, when coupled with the selection of clones with increasing capacity for autonomous growth, results the multi-step conversion of normal cells to a malignant state (42, 43). Most cancers are caused by exposure to carcinogens present in the environment or produced by cellular metabolism, often influenced by specific life styles 44-46). However, it has become increasingly clear that cancer cells contain extensively altered genomes, widely attributed to an intrinsic state of genomic instability (47-49). Specific genes required to maintain genome integrity and that also function to prevent cancer have been identified in humans with familial cancer syndromes and in mouse knockout models. These include genes involved in recombination, DNA repair, mitotic spindle checkpoint control and cell cycle regulation (50-54). Since genomic instability can clearly drive carcinogenesis, presumably by enhancing the likelihood of mutations in oncogenes and tumor suppressor genes and since chromosome alterations appear to have greater genetic impact than the accumulation of point mutations, genomic instability has been proposed to play a greater role in carcinogenesis than somatic mutations (48, 55-57). However, the origin of most genetic alterations in human cancer cells has not been established; hence, the relative importance of somatic mutations and genomic instability in carcinogenesis remains an active area of controversy (48, 56-58).

Allelic imbalance and losses of heterozygosity (LOH) are the most common genetic alterations in human cancers, which may harbor over 10,000 regions of LOH per cell (37, 59-60). LOH contributes to carcinogenesis by altering the dosage of genetically and epigenetically modified genes (62). These include over 60 characterized recessive cancer genes (tumor suppressors) (63) and other alleles that may enhance cell fitness. While mutations in genes required for genome maintenance can produce high levels of LOH, except for a subset of tumors with microsatellite instabilities or associated with inherited cancer susceptibility syndromes most tumors appear to lack caretaker gene mutations (47, 55-57). Extensive LOH has been observed in non malignant lesions—in some cases at levels comparable to those of invasive tumors (37, 59-61). Thus, genomic instability could be an early event in carcinogenesis. Alternatively, stem cells in the surrounding normal tissues could have equally high levels of LOH that escape detection because, in the absence of clonal growth, sufficiently pure cell populations are not available for analysis (64).

It has been argued that normal mutation rates are not sufficient to account for the levels of genetic alterations found in cancers (48), and alternatively that the prevalence of mutations is no higher than would be expected to accumulate in the stem cells assuming many rounds of cell division (64). The issue is complicated by the possibility that stem cells may posses specialized mechanisms to suppress mutations, possibly as a defense against oncogenic transformation (65-69). Clearly, a better understanding of the origins of LOH will influence opinion about the relative roles of somatic mutations and genomic instability in carcinogenesis. Therefore, the following question was addressed: to what extent are carcinogens, including agents commonly known to induce point mutations, capable of inducing genome-wide LOH in normal diploid stem cells?

To answer this question, genes tagged by a gene trap retrovirus were used to quantify carcinogen-induced LOH at 53 sites in the genome of normal embryonic stem (ES) cells. The entrapment clones were biologically normal as assessed by their ability to produce germline chimeras and normal offspring, and thus lacked coincidental mutations affecting genome stability. By quantifying the frequencies of LOH at many sites in the genome, this invention provides the first genome-wide analysis of carcinogen-induced LOH in any mammalian cell type. Finally, the use of ES cells permitted direct comparisons between the effects of chemical carcinogens and the Bloom's syndrome mutation, a well-characterized mutator phenotype that has also been analyzed in genetically-deficient ES cells (7, 38, 70). The present invention shows that limited exposure to a variety of carcinogens induces genome-wide LOH at per-gene frequencies approaching one percent. In short, the carcinogens produced the appearance of chromosomal instability in normal stem cells in the absence of a genetically activated genomic instability phenotype.

### Carcinogen-Induced LOH

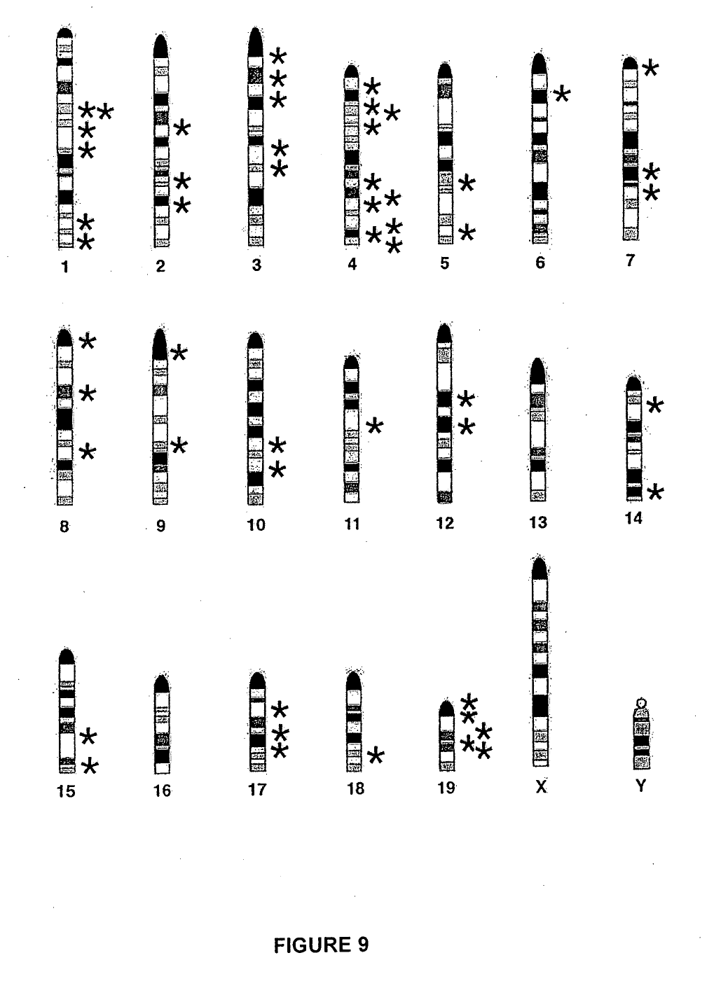

Carcinogen-induced LOH was measured in a panel of 53 mouse embryonic stem cell clones, each containing a neomycin resistance gene (Neo) inserted into a different cellular gene (FIG. 9) by the GTR1.3 gene trap retrovirus. Gene entrapment by GTR1.3 involves selection for inserted Neo sequences that can splice to the 3′ ends of cellular genes (Lin et al.,). The disrupted genes were identified by sequencing Neo-gene fusion transcripts and were localized on the mouse genome. Previous studies have shown that cells homozygous for GTR1.3-induced mutations can be selected from heterozygous cells simply by selecting for resistance to higher concentrations of G418, a method first shown to select for homozygous mutations induced by gene targeting (15). Mitotic recombination appears to be the preferred mechanism of spontaneous LOH involving Neo genes inserted in ES cells (17) and LOH involving other genes and cell types in vivo (12, 13). LOH doubles the number of Neo genes per cell and thus allows moderately resistant cells to acquire resistance to higher concentrations of G418. The frequencies of spontaneous LOH measured at the 53 different sites ranged from 1.3×10⁻⁵ to 1.2×10⁻⁴ (Table 3), similar to those reported for other inserted neomycin resistance genes (15, 17) in ES cells and for loci such as TK and APRT in other cell types (12, 13).

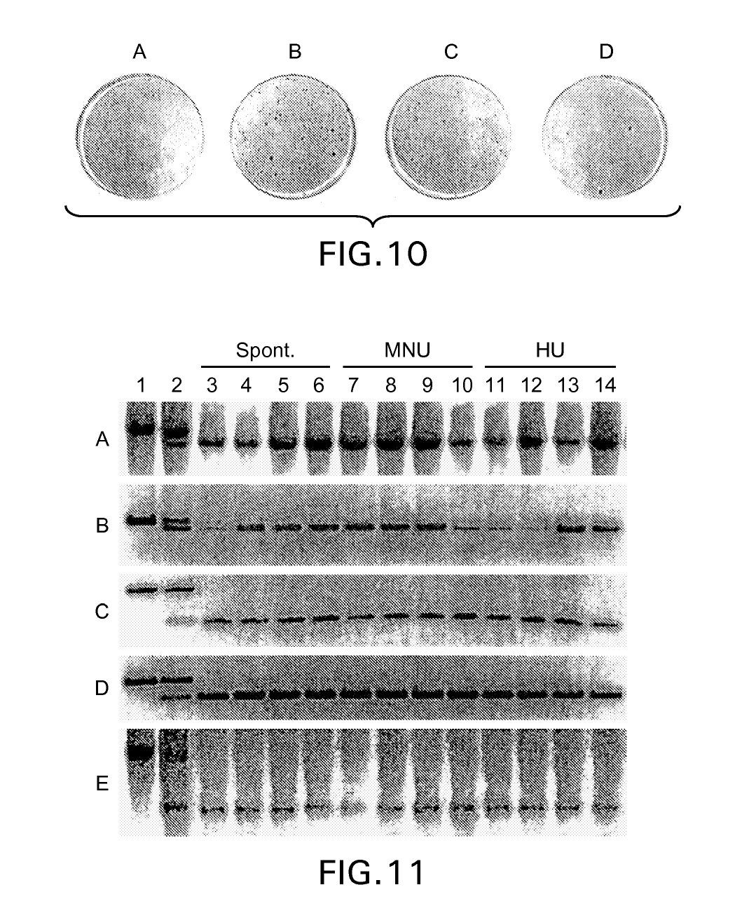

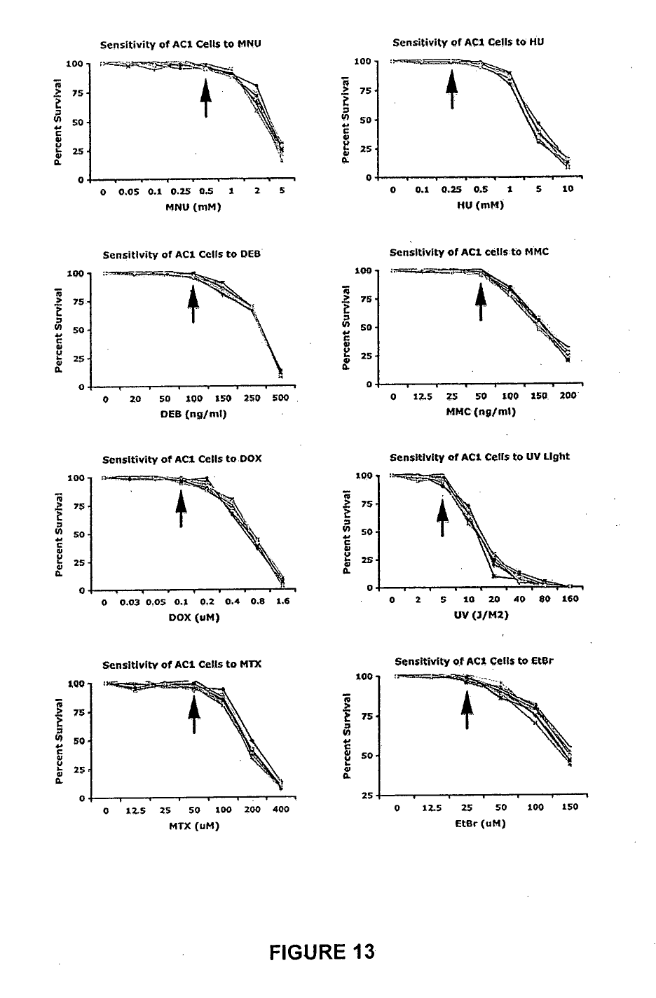

A variety of chemical agents were tested for their ability to enhance the frequencies at which mutant cells survive in 2.0 mg/ml G418. Methyl-nitrosurea (MNU) is an alkylating agent that produces a variety of mono-methylated DNA adducts, hydroxyurea (HU) stalls DNA replication complexes, doxorubicin interferes with DNA synthesis, methothrexate is a competitive inhibitor of dihydrofolate reductase but is not genotoxic, diepoxybutane and mitomycin C induce inter-strand DNA crosslinks, UV irradiation causes intra- and inter-strand pyrimidine dimmers, and ethidium bromide intercalates between DNA strands to damage DNA (For additional information about these agents see http://toxnet.nlm.nih.gov and http://lisntweb.swan.ac.uk/cmgt/index.htm). Treatment with 0.5 mM MNU or 0.25 mM HU dramatically increased the number of colonies surviving in high G418 (FIG. 10). The fold-increase for MNU and HU ranged from 39-123 and 18-68, respectively (Table 3). The optimal concentrations of each agent to stimulate colony formation with minimal toxicity (<5% loss of cell viability) were determined in advance (FIG. 13). Other genotoxic agents that have been reported to promote recombination doxorubicin (0.1 μM), diepoxybutane (100 ng/mL), mitomycin C (50 ng/mL) and ultraviolet light (5 J/m²) also enhanced colony formation by an average of 15, 16, 14, and 10-fold. However, ethidium bromide (25 μg/ml) and methothrexate (50 μM) had no significant effect (Table 3). Each of these agents was tested two or more times on at least 25 entrapment lines.

Genotypic analysis of 5 different mutants confirmed that 100% of colonies that survived high G418 selection following carcinogen treatment had undergone LOH (FIG. 11) compared to 85% of spontaneously resistant colonies. Thus, colony formation in 2.0 mg/ml G418 provided a direct measure of carcinogen-induced LOH at each entrapment locus. The overall extent of LOH induced by a single exposure to non-toxic levels of either MNU or HU was remarkably high (Table 3), approaching 1% of the genome.

### LOH is a Transient Response to Carcinogens

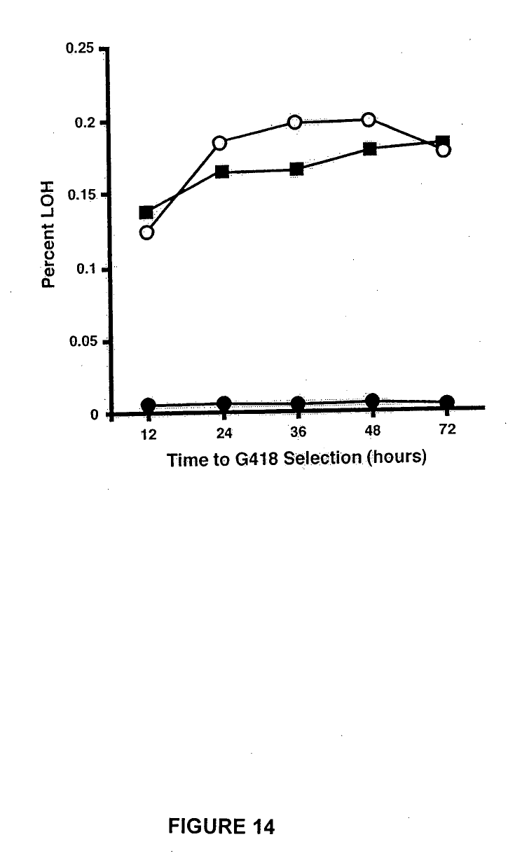

Two types of experiments were performed to assess whether frequencies of LOH were transiently or stably elevated following carcinogen exposure. First, cells were treated with MNU and HU as before and the percentages of cells having undergone LOH were determined by selection in 2.0 mg/ml G418 at various times thereafter. Over 90% of the total LOH was induced within 24 hours of MNU and HU exposure, and only minimal additional LOH occurred subsequently (FIG. 14). Second, it was asked whether LOH frequencies at a second locus were elevated in cells having undergone LOH at the entrapment locus. For this, a Herpes Simplex Virus thymidine kinase (TK) gene was introduced into cells containing an entrapment allele of the Hesx1 gene and frequencies of TK gene loss were measured by selection in gangcyclovir. These studies utilized the cell line containing the TK gene (C8TK1) and derivatives of C8TK1 that had undergone LOH at the entrapment locus either spontaneously (C8TK1sN) or after treatment with either MNU (C8TKmN) or HU (C8TK1hN). As shown in Table 2, the frequencies of spontaneous TK gene loss were similar in all cells regardless of whether carcinogens had been used previously to induce LOH at the entrapment locus. Moreover, the stability of the TK gene following carcinogen treatment was largely unaffected by prior selection for LOH involving the entrapment locus. Similar results were also obtained with a second TK-containing line (C8TK2, Table 4). Together these experiments indicate that carcinogen-induced LOH results from an acute response rather than from a stably altered cellular phenotype.

### Effect of Chromosome Position on Carcinogen-Induced LOH

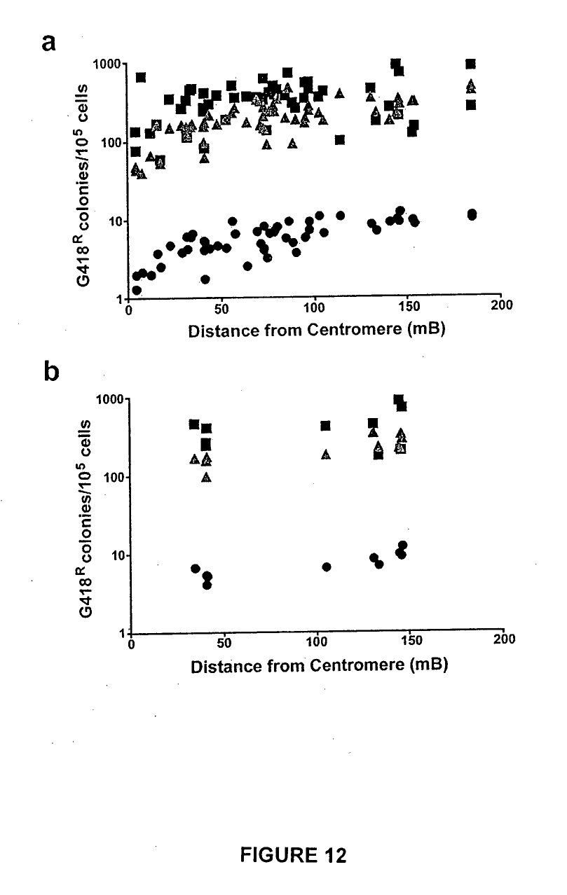

The frequency of spontaneous colony formation in high G418 was previously reported to increase with increasing distance from the centromere (Lin et al), consistent with previous studies suggesting that mitotic recombination plays a significant role in spontaneous losses of heterozygosity (34). Similar chromosome position effects were also observed (FIG. 12) following treatment with HU but not MNU (for example, R² values for loci on chromosome 4 were 0.65 and 0.11 following HU and MNU treatment, respectively) suggesting that mechanisms other than mitotic recombination (e.g. gene conversion) were responsible for most of the MNU-induced LOH, consistent with studies in mouse lymphocytes (34). However, the ES cells used in the present study were derived from inbred mice and are naturally homozygous at all loci, and thus cannot be used to distinguish among the possible mechanisms for generating LOH.

### Gene Entrapment in Studies of Genome-Wide LOH

Entrapment ES cell clones provide an important in vitro model to study spontaneous and chemically induced LOH. ES cells are representative of self-renewing stem cells that serve as the precursors to cancer (38), and their use in mutagenesis studies is potentially important since stem cells may posses specialized mechanisms to suppress mutations as a defence against oncogenic transformation (25-29). The clones are biologically normal as assessed by their ability to produce germline chimeras (10 of 10 clones tested) and normal offspring and thus lack coincidental mutations that might affect genome maintenance. Libraries of entrapment clones characterized for mouse genome mutagenesis provide large numbers of genetic markers that for the first time allow LOH frequencies to be measured at many sites in the genome. Rates of spontaneous LOH observed in ES cells are similar to those reported in a variety of other mammalian cell types (39, 40). Moreover, the influence of chromosome position indicates that the rates of spontaneous and HU-induced LOH do not primarily reflect localized effects of the integrated gene trap vector.

The use of entrapment ES cells also permits direct comparisons between the effects of chemical carcinogens and specific DNA repair defects such as the Bloom's syndrome mutation. Given the ease of creating defined mutations that can be transferred back and forth between ES cells and mice, ES cells provide an ideal system to compare the effects of different mutations on spontaneous and carcinogen induced-LOH in a normal, and potentially isogenic cellular background. Whether endogenous or exogenous carcinogens contribute to genome-wide changes associated with defects in genome maintenance can be tested. For example, mice expressing reduced levels of Bub1B, a protein involved in mitotic spindle checkpoint control, form tumors only after carcinogen exposure (41). It can also be possible to assess how specific DNA repair/genome maintenance pathways influence the types of recombination events induced by different genotoxic agents (37).

The GTR1.3 vector has features that allow the selection of homozygous mutant cells except in cases where gene entrapment disrupts genes required for cell growth or viability. The present invention shows that MNU and HU can be used to enhance the recovery of clones homozygous for recessive mutations during phenotype-based genetic screens in mammalian cells (31, 32, 42). The vector can also allow mutagenesis screens to be carried out in a greater variety of cell backgrounds.

### LOH as a Somatic Mutation: Implications for the Carcinogenicity of Mutagens Like MNU

Although mutagens such as MNU have been reported to induce LOH (39, 43-48)., these studies utilized non-mammalian systems or tumor-derived cell lines or were limited to only one or two loci. The present invention provides the first genome-wide analysis of carcinogen-induced LOH in any mammalian cell type and the first analysis involving normal diploid stem cells. As with most laboratory assessments of carcinogen risk, it is difficult to extrapolate from the concentrations of carcinogen used experimentally to the levels of exposure in human populations that typically occur over several decades. However, carcinogen concentrations were minimally toxic and were similar to those commonly used to induce tumors in animals.

LOH contributes to carcinogenesis by altering the dosage of genetically and epigenetically modified genes (22), including recessive cancer genes (tumor suppressors) of which over 60 have been characterized (23). The ability of MNU and other agents used in the present study to induce point mutations is well established. These agents are also clastogens as assessed by their ability to induce chromosome aberrations and sister-chromatid exchanges (http://toxnet.nlm.nih.gov). The results presented herein indicate that the induction of LOH by a variety of mutagens occurs in normal stem cells at frequencies 2-4 orders of magnitude higher on a per-gene basis than the reported induction of point mutations. This could contribute to the notion that chromosome alterations such as LOH appear to have a greater impact on tumor cell genomes than the accumulation of point mutations (7, 14-16).

Frequencies of carcinogen-induced LOH were higher in some cases than the reported rates of LOH observed in ES cells homozygous for a mutation in the Bloom syndrome gene (Blm) (30, 31), an inherited DNA repair defect that results in greatly increased risk of cancer. Higher LOH frequencies were observed even allowing for differences in plating efficiencies of entrapment clones (10-50%, data not shown) as compared to the Blm-deficient ES cells (30%) (31). In short, the carcinogens tested produced the appearance of chromosome instability in normal stem cells in the absence of a genetically determined mutator phenotype. Of course, the Blm mutation causes a persistent state of chromosomal instability, whereas, the rates of carcinogen-induced LOH are elevated only transiently following carcinogen exposure. While it is not clear how certain mutations affecting DNA repair induce LOH, it would appear that certain types of adducted DNA and/or stalled replication complexes can promote LOH regardless of whether they are caused directly by genotoxic agents or indirectly by genetic attenuation of DNA repair pathways. Just as the carcinogenicity of the Blm mutation has been attributed to the induction of LOH, the carcinogenicity of a variety of mutagens may result as much from their ability to induce LOH as from their ability to induce point mutations.

### LOH as Somatic Mutation: Implications Regarding the Origins of LOH in Human Cancer

Extensive LOH in cancer cells is widely assumed to result from chromosomal instability; however, this conclusion is almost always based on the prevalence of LOH rather than on actual rate measurements (6). The present invention shows that extensive LOH is induced in normal stem cells as an acute response to non-toxic levels of various carcinogens. Therefore, it is possible that much of the LOH observed in non-hereditary cancers could result from prior exposure to genotoxic agents rather than from a state of genomic instability during the carcinogenic process. This is consistent with the fact that over 80% of cancers are caused by carcinogens present in the environment or produced by cellular metabolism (3-5), explains the apparent absence of mutations in genes required for DNA repair/genome maintenance in most cancers (6, 15, 16) and may account for the high levels LOH reported in several types of non cancerous lesions (18-21).

In summary, the present invention describes the first mechanism capable of generating high levels of LOH in the absence of a genetically activated chromosomal instability phenotype. Intrinsically low mutation rates and apoptosis in self-renewing stem cells have been proposed as mechanisms to suppress carcinogenesis (25-29). Similarly, the efficient use of sequences from homologous chromosomes to repair DNA damage and/or resolve stalled replication complexes could function to prevent coding sequences mutations. However, the process causes extensive losses of heterozygosity with the likely consequence of unmasking recessive mutations in tumor suppressor genes.

### Cell Culture

The AC1 embryonic stem cell line was derived from 3.5d blastocysts from 129svJ mice. AC1 cells were infected with the GTR1.3 poly(A) gene trap vector and entrapment clones were isolated in 300 μg/ml G418. GTR1.3 inserts a neomycin phosphotransferase gene (Neo) expressed from the constitutive Pol2 gene promoter. Selection for neomycin (G418) resistance generates cell clones in which the Neo gene splices to 3′ exons of cellular genes Genes disrupted in the entrapment clones were identified by sequencing cellular sequences appended to Neo fusion transcripts. ES cells were maintained at 37° in DMEM supplemented with 15% fetal bovine serum, non-essential amino acids, L-glutamine, β-mercaptoethanol, and LIF.

### Colony Selection and Chemical Treatment of Cell

Serially diluted cells were plated onto 150 mm plates containing drug-free media and allowed to attach overnight. Unattached cells were removed and media containing the indicated concentrations of methyl-nitrosourea, hydroxyurea, ethidium bromide, doxorubicin, methotrexate, diepoxybutane, or mitomycin C was put onto cells for 4 hours (or cells were exposed to ultraviolet light in the absence of media and allowed to recover in drug-free media for 4 hours). Cells were then rinsed twice with drug-free media and selection media containing 0.0, 0.3, or 2.0 mg/ml G418 was put onto cells. After 12 days of selection, the number of colonies surviving was counted and the frequency of colony formation was determined by dividing the number of colonies obtained from 2.0 mg/ml G418 selection to that obtained from parallel experiments with 0.3 mg/ml G418 selection. TK gene loss was assessed following selection in media containing 2 μg/ml gancyclovir.

### Genotypic Analysis of LOH

Genotypic analysis was performed by Southern blotting and PCR. Southern blot analysis was performed on 5 μg genomic DNA that had been digested with a restriction enzyme and resolved on 0.9% agarose gels. Southern blot hybridization was performed using DNA probes obtained by PCR amplification of genomic DNA adjacent to the site of retroviral vector insertion. PCR analysis was performed on 200 ng of genomic DNA with three primers. The first primer was in the sense orientation and was specific for genomic DNA 5′ to the site of retroviral vector insertion. Two additional primers were added that were in the antisense orientation-one was specific for sequence 3′ of the retroviral vector insertion and the other specific for the LTR portion of the retroviral vector insertion. Using these three primers, PCR amplification of genomic DNA yielded a smaller DNA fragment when the entrapment vector was present and a larger DNA fragment when the entrapment vector was absent.

TABLE 2

| | Inducer | | | | | |
|---|---|---|---|---|---|---|
| | EL | | | Freq. | | |
| Clone | LOH | Genotype | Treatment | TK loss | | Ratio |
| C8TK1 | none | TK⁺ EL^{+/-} | none | 1.9 × 10⁻⁵ | | |
| | | | | | {close oversize brace} | 2.8 |
| C8TK1sN | Spont. | TK⁺ EL^{-/-} | none | 5.3 × 10⁻⁵ | | |
| C8TK1mN | MNU | TK⁺ EL^{-/-} | none | 1.4 × 10⁻⁵ | | |
| | | | | | {close oversize brace} | 2.6 |
| C8TK1sN | Spont. | TK⁺ EL^{-/-} | none | 5.3 × 10⁻⁵ | | |
| | | | | | {close oversize brace} | 0.6 |
| C8TK1hN | HU | TK⁺ EL^{-/-} | none | 8.4 × 10⁻⁵ | | |
| C8TK1sN | Spont. | TK⁺ EL^{-/-} | HU | 2.3 × 10⁻⁴ | | |
| | | | | | {close oversize brace} | 1.7 |
| C8TK1hN | HU | TK⁺ EL^{-/-} | HU | 3.9 × 10⁻⁴ | | |
| C8TK1sN | Spont. | TK⁺ EL^{-/-} | MNU | 2.6 × 10⁻⁴ | | |
| | | | | | {close oversize brace} | 1.8 |
| C8TK1mN | MNU | TK⁺ EL^{-/-} | MNU | 4.7 × 10⁻⁴ | | |

Table 2. Carcinogen-induced LOH does not result from a stably altered cellular phenotype. The HSV thymidine kinase (TK) gene was introduced into cells containing an entrapment mutation in the Hesx1 gene. A TK expressing (TK⁺) clone (C8TK1) was used to select for cells that had undergone LOH at the entrapment locus (EL) spontaneously (C8TK1sN) or following treatment with HU (C8TK1hN) or MNU (C8TK1mN). The frequencies of TK gene loss were compared in cells with and without prior selection for LOH at the entrapment locus and the differences were expressed as the indicated ratios.

Table 3. The frequency of colony formation in high concentrations of G418. The frequency of colony formation is listed for all 53 clones examined. Frequency was determined by dividing the number of colonies surviving 2.0 mg/ml G418 selection to the number surviving parallel experiments with 0.3 mg/ml G418 selection, and is the average of at least 3 independent experiments for each clone. Standard deviations, which ranged from 5 to 70% of the mean values, have been omitted for clarity. The Student's t-test was used to determine statistical significance of differences between treated and the “2.0 mg/ml G418 only” condition, *p<0.05, **p<0.01. Genes disrupted by gene entrapment in each clone are indicated when known. The sequences of fusion transcripts cloned by 3′RACE have been submitted to the Genbank GSS database, and the accession number of each sequence is listed. The chromosomal location of each entrapment vector was determined from BlastN matches between fusion transcripts and mouse genome sequences.

TABLE 3

| Clone | GSS | 2 mg/ml G418 | 2 mg/ml G418 | 2 mg/ml G418 | 2 mg/ml G418 | Chromosome | Gene Disrupted |
|---|---|---|---|---|---|---|---|
| ID | Accession # | only | 0.5 mM MNU | 0.25 mM HU | 25 mM EtBr | Location | and Gene ID or MGI |
| b3p3-d8 | CZ169573 | 2.53 × 10-5 | 3.63 × 10-3** | 1.73 × 10-3** | 3.17 × 10-5 | 1C2 | ND |
| | | | (+123-fold) | (+68-fold) | | | |
| b3p3-d4 | CZ169572 | 3.20 × 10-5 | 1.36 × 10-3** | 9.02 × 10-4* | 3.15 × 10-5 | 1C3 | LOC227288 (2448715) |
| | | | (+43-fold) | (+28-fold) | | | |
| b3p4-d12 | CZ169762 | 4.05 × 10-5 | 3.32 × 10-3** | 2.06 × 10-3* | 2.70 × 10-5 | 1C3 | (MGI: 104517) |
| | | | (+82-fold) | (+51-fold) | | | |
| b3p4-g3 | CZ169810 | 4.28 × 10-5 | 3.48 × 10-3** | 1.45 × 10-3* | 3.75 × 10-5 | 1C3 | Xrcc5 (MGI: 104517) |
| | | | (+81-fold) | (+34-fold) | | | |
| b2p1-f12 | CZ169705 | 1.09 × 10-4 | 8.44 × 10-3** | 2.72 × 10-3* | 1.01 × 10-4 | 1H5 | Bnpt1 (MGI: 1338800) |
| | | | (+77-fold) | (+25-fold) | | | |
| b2p1-h5 | CZ169717 | 1.01 × 10-4 | 7.59 × 10-3** | 4.21 × 10-3* | 1.29 × 10-4 | 1H5 | ND |
| | | | (+75-fold) | (+42-fold) | | | |
| b3p4-g9 | CZ169804 | 4.91 × 10-5 | 3.17 × 10-3** | 1.59 × 10-3* | 4.10 × 10-5 | 2C2 | Grb14 (Mm.33806) |
| | | | (+65-fold) | (+32-fold) | | | |
| b3p4-e1 | CZ169763 | 9.59 × 10-5 | 6.22 × 10-3** | 3.03 × 10-3* | 8.85 × 10-5 | 2G3 | KIF3B (MGI: 107688) |
| | | | (+65-fold) | (+32-fold) | | | |
| b2p1-a9 | CZ169685 | 8.53 × 10-5 | 4.49 × 10-3** | 3.05 × 10-3* | 9.22 × 10-5 | 2H1 | Cdk5rap1 (MGI: 1914221) |
| | | | (+53-fold) | (+36-fold) | | | |
| b3p4-b12 | CZ169783 | 9.59 × 10-5 | 4.98 × 10-3** | 2.28 × 10-3* | 8.60 × 10-5 | 3A1 | ND |
| | | | (+52-fold) | (+24-fold) | | | |
| b3p4-b1 | CZ169784 | 3.73 × 10-5 | 1.68 × 10-3** | 1.64 × 10-3* | 4.40 × 10-5 | 3A2 | S12207 hypothetical protein |
| | | | (+45-fold) | (+44-fold) | | | |
| b3p4-a5 | CZ169854 | 1.74 × 10-5 | 8.13 × 10-4* | 6.19 × 10-4* | 2.51 × 10-5 | 3B | ND |
| | | | (+47-fold) | (+36-fold) | | | |
| b3p3-b6 | CZ169662 | 5.00 × 10-5 | 3.00 × 10-3** | 9.18 × 10-4* | 4.69 × 10-5 | 3F1 | ND |
| | | | (+60-fold) | (+18-fold) | | | |
| b2p1-f10 | CZ169711 | 5.87 × 10-5 | 5.37 × 10-3** | 1.94 × 10-3* | 7.23 × 10-5 | 3F2.1 | Prune (MGI: 1925152) |
| | | | (+91-fold) | (+33-fold) | | | |
| b3p4-a1 | CZ169780 | 6.67 × 10-5 | 4.60 × 10-3** | 1.66 × 10-3* | 5.95 × 10-5 | 4A4 | 1810030N24Rik |
| | | | (+69-fold) | (+25-fold) | | | (MGI: 1913541) |
| b3p3-a4 | CZ169660 | 5.20 × 10-5 | 4.05 × 10-3** | 1.73 × 10-3* | 7.30 × 10-5 | 4A5 | (Mm.96573) |
| | | | (+78-fold) | (+33-fold) | | | |
| b3p3-a9 | CZ169623 | 5.37 × 10-5 | 2.65 × 10-3** | 9.70 × 10-4* | 5.48 × 10-5 | 4A5 | (MGI: 3045357) |
| | | | (+49-fold) | (+18-fold) | | | |
| b2p1-b9 | CZ169682 | 4.07 × 10-5 | 2.41 × 10-3** | 1.55 × 10-3* | 5.11 × 10-5 | 4B1 | Spink4 (MGI: 1341848) |
| | | | (+59-fold) | (+38-fold) | | | |
| b3p4-b8 | CZ169787 | 6.60 × 10-5 | 4.18 × 10-3** | 1.80 × 10-3* | 5.60 × 10-5 | 4C6 | ND |
| | | | (+63-fold) | (+27-fold) | | | |
| b2p1-d8 | CZ170502 | 8.48 × 10-5 | 4.42 × 10-3** | 3.43 × 10-3* | 1.19 × 10-4 | 4D2.3 | Smpdl3b (MGI: 1916022) |
| | | | (+52-fold) | (+40-fold) | | | |
| b2p1-e3 | CZ169699 | 6.97 × 10-5 | 3.74 × 10-3** | 2.25 × 10-3* | 6.56 × 10-5 | 4D2.3 | 4930555I21Rik |
| | | | (+54-fold) | (+32-fold) | | | (MGI: 1926056) |
| b3p3-g10 | CZ169605 | 1.22 × 10-4 | 7.08 × 10-3** | 2.86 × 10-3* | 2.15 × 10-4 | 4E1 | ND |
| | | | (+58-fold) | (+24-fold) | | | |
| b3p4-f2 | CZ169770 | 9.20 × 10-5 | 5.04 × 10-3** | 3.31 × 10-3* | 1.57 × 10-4 | 4E1 | Mad212 (MGI: 1919140) |
| | | | (+55-fold) | (+36-fold) | | | |
| b3p3-d9 | CZ169663 | 9.84 × 10-5 | 8.77 × 10-3** | 2.21 × 10-3* | 9.73 × 10-5 | 4E2 | D4Cole1e gene |
| | | | (+89-fold) | (+22-fold) | | | |
| b3p3-h4 | CZ169643 | 3.71 × 10-5 | 2.53 × 10-3** | 1.84 × 10-3** | 4.62 × 10-5 | 5E2 | D430040L24Rik |
| | | | (+70-fold) | (+50-fold) | | | (MGI: 2444469) |
| b3p3-c12 | CZ169567 | 8.97 × 10-5 | 2.60 × 10-3* | 1.76 × 10-3* | 5.19 × 10-5 | 5G2 | D130017N08Rik |
| | | | (+29-fold) | (+20-fold) | | | (MGI: 2443273) |
| b2p1-h8 | CZ169725 | 3.84 × 10-5 | 2.60 × 10-3** | 1.61 × 10-3** | 6.40 × 10-5 | 6A3.3 | Atp6v1f (MGI: 1913394) |
| | | | (+68-fold) | (+42-fold) | | | |
| b2p1-h1 | CZ169726 | 2.52 × 10-5 | 1.59 × 10-3** | 5.32 × 10-4* | 8.33 × 10-5 | 7A2 | Ech1 (MGI: 1858208) |
| | | | (+63-fold) | (+21-fold) | | | |
| b3p4-f6 | CZ169773 | 9.33 × 10-5 | 4.46 × 10-3** | 2.41 × 10-3* | 7.34 × 10-5 | 7E3 | 1600010M07 Rik |
| | | | (+48-fold) | (+26-fold) | | | (MGI: 1917031) |
| b2p1-d9 | CZ169702 | 7.25 × 10-5 | 5.46 × 10-3** | 2.76 × 10-3* | 8.25 × 10-5 | 7E3 | 1600010M07Rik (MGI: 191) |
| | | | (+75-fold) | (+38-fold) | | | |
| b3p4-h11 | CZ169856 | 2.00 × 10-5 | 1.30 × 10-3** | 6.64 × 10-4* | 1.96 × 10-5 | 8A1.1 | 4933439N14Rik |
| | | | (+65-fold) | (+33-fold) | | | (Mm.160052) |
| b3p4-f10 | CZ169812 | 4.64 × 10-5 | 3.78 × 10-3** | 1.65 × 10-3* | 1.81 × 10-5 | 8A4 | ND |
| | | | (+81-fold) | (+36-fold) | | | |
| b3p4-c2 | CZ169794 | 9.34 × 10-5 | 7.13 × 10-3** | 4.67 × 10-3** | 7.31 × 10-5 | 8C3 | (Mm.24524) |
| | | | (+76-fold) | (+50-fold) | | | |
| b2p1-d10 | CZ169712 | 2.14 × 10-5 | 1.62 × 10-3** | 3.98 × 10-4* | 3.87 × 10-5 | 9A1 | Nr1b1 (MGI: 97856) |
| | | | (+76-fold) | (+19-fold) | | | |
| b2p1-b4 | CZ169688 | 7.26 × 10-5 | 3.84 × 10-3** | 2.35 × 10-3* | 9.06 × 10-5 | 9E1 | Dppa5 (MGI: 101800) |
| | | | (+53-fold) | (+32-fold) | | | |
| b3p4-g8 | CZ169802 | 5.71 × 10-5 | 3.70 × 10-3** | 1.93 × 10-3* | 1.00 × 10-4 | 10C1 | Rfx4 (MGI: 1918387) |
| | | | (+75-fold) | (+34-fold) | | | |
| b3p3-g6 | CZ169601 | 5.78 × 10-5 | 3.42 × 10-3** | 1.68 × 10-3* | 2.86 × 10-5 | 10C2 | Cradd (MGI: 1336168) |
| | | | (+59-fold) | (+29-fold) | | | |
| b3p4-b2 | CZ169785 | 6.53 × 10-5 | 3.54 × 10-3** | 2.60 × 10-3* | 7.70 × 10-5 | 11B1.3 | 2010001A14Rik |
| | | | (+54-fold) | (+40-fold) | | | (MGI: 1923766) |
| b3p4-e7 | CZ170247 | 4.33 × 10-5 | 1.86 × 10-3** | 1.89 × 10-3* | 3.40 × 10-5 | 12C1 | Mipol1 (MGI: 1920740) |
| | | | (+43-fold) | (+44-fold) | | | |
| b2p1-a5 | CZ169683 | 6.62 × 10-5 | 4.06 × 10-3** | 3.24 × 10-3* | 6.27 × 10-5 | 12C3 | Galntl1 (MGI: 1917754) |
| | | | (+61-fold) | (+35-fold) | | | |
| b3p3-c8 | CZ169557 | 4.72 × 10-5 | 3.45 × 10-3** | 1.50 × 10-3* | 6.36 × 10-5 | 14A3 | Hesx1 gene (MGI: 96071) |
| | | | (+73-fold) | (+32-fold) | | | |
| b3p4-d10 | CZ169761 | 1.08 × 10-4 | 9.49 × 10-3** | 5.83 × 10-3** | 1.08 × 10-4 | 14E5 | Phgdhl1 (MGI: 1916139) |
| | | | (+88-fold) | (+54-fold) | | | |
| b3p4-e3 | CZ169766 | 6.85 × 10-5 | 4.97 × 10-3** | 2.78 × 10-3* | 7.87 × 10-5 | 15D3 | ND |
| | | | (+73-fold) | (+41-fold) | | | |
| b3p4-c9 | CZ169841 | 1.10 × 10-4 | 3.54 × 10-3* | 2.23 × 10-3* | 1.07 × 10-4 | 15F2 | ND |
| | | | (+39-fold) | (+20-fold) | | | |
| b3p4-c1 | CZ169790 | 4.29 × 10-5 | 2.94 × 10-3** | 2.14 × 10-3** | 5.75 × 10-5 | 17B3 | LOC433110 (Mm.45676) |
| | | | (+69-fold) | (+50-fold) | | | |
| b2p1-f3 | CZ169708 | 7.00 × 10-5 | 3.57 × 10-3** | 3.30 × 10-3** | 8.80 × 10-5 | 17E1.3 | Dlgap1 (MGI: 1346065) |
| | | | (+51-fold) | (+47-fold) | | | |
| b3p4-c12 | CZ169852 | 8.20 × 10-5 | 6.05 × 10-3** | 2.69 × 10-3* | 8.89 × 10-5 | 17E2 | ND |
| | | | (+74-fold) | (+33-fold) | | | |
| b3p1-h1 | CZ169622 | 8.02 × 10-5 | 4.44 × 10-3** | 3.36 × 10-3* | 3.17 × 10-5 | 18E2 | 4933427L07Rik |
| | | | (+55-fold) | (+42-fold) | | | (MGI: 1918480) |
| b3p3-h8 | CZ169641 | 1.31 × 10-5 | 1.37 × 10-3** | 4.08 × 10-4* | 2.05 × 10-5 | 19A | RBM4 (MGI: 1100865) |
| | | | (+105-fold) | (+37-fold) | | | |
| b2p1-c6 | CZ169692 | 1.98 × 10-5 | 7.72 × 10-4* | 4.34 × 10-4* | 1.18 × 10-5 | 19A | ND |
| | | | (+39-fold) | (+22-fold) | | | |
| b2p1-e10 | CZ169707 | 6.11 × 10-5 | 3.28 × 10-3** | 1.52 × 10-3* | 1.08 × 10-4 | 19C1 | ND |
| | | | (+54-fold) | (+25-fold) | | | |
| b2p1-d3 | CZ169695 | 4.23 × 10-5 | 1.73 × 10-3** | 1.20 × 10-3* | 6.50 × 10-5 | 19C1 | ND |
| | | | (+41-fold) | (+28-fold) | | | |
| b3p3-h2 | CZ170481 | 6.02 × 10-5 | 4.40 × 10-3** | 1.54 × 10-3* | 7.04 × 10-5 | 19C1 | AW210596 (MGI: 2147718) |
| | | | (+73-fold) | (+26-fold) | | | |

*p < 0.05
**p < 0.01
ND = no data

Table 4. Carcinogen-induced LOH does generate genetically unstable cells. The HSV thymidine kinase (TK) gene was introduced into cells containing an entrapment mutation in the Hesx1 gene. A TK expressing (TK+) clone (C8TK2) was used to select for cells that had undergone LOH at the entrapment locus (EL) spontaneously (C8TK2sN) or following treatment with HU (C8TK2hN) or MNU (C8TK2mN). The frequencies of spontaneous TK gene loss (Treatment=none) or TK gene loss following treatment with HU or MNU were compared in cells with (EL^{-/-}) and without prior selection for LOH (EL^{+/+}) at the entrapment locus and the differences were expressed as the indicated ratios.

TABLE 4

| | Inducer | | | | | |
|---|---|---|---|---|---|---|
| | EL | | | Freq. | | |
| Clone | LOH | Genotype | Treatment | TK loss | | Ratio |
| C8TK1 | none | TK⁺ EL^{+/-} | none | 1.9 × 10⁻⁵ | | |
| | | | | | {close oversize brace} | 2.7 |
| C8TK1sN | Spont. | TK⁺ EL^{-/-} | none | 4.7 × 10⁻⁵ | | |
| C8TK1mN | MNU | TK⁺ EL^{-/-} | none | 1.3 × 10⁻⁵ | | |
| | | | | | {close oversize brace} | 2.4 |
| C8TK1sN | Spont. | TK⁺ EL^{-/-} | none | 5.3 × 10⁻⁵ | | |
| | | | | | {close oversize brace} | 2.3 |
| C8TK1hN | HU | TK⁺ EL^{-/-} | none | 1.2 × 10⁻⁵ | | |
| C8TK1sN | Spont. | TK⁺ EL^{-/-} | HU | 2.8 × 10⁻⁴ | | |
| | | | | | {close oversize brace} | 1.8 |
| C8TK1hN | HU | TK⁺ EL^{-/-} | HU | 4.9 × 10⁻⁴ | | |
| C8TK1sN | Spont. | TK⁺ EL^{-/-} | MNU | 2.9 × 10⁻⁴ | | |
| | | | | | {close oversize brace} | 1.6 |
| C8TK1mN | MNU | TK⁺ EL^{-/-} | MNU | 4.5 × 10⁻⁴ | | |

Throughout this application, various publications are referenced. The disclosures of these publications in their entireties are hereby incorporated by reference into this application in order to more fully describe the state of the art to which this invention pertains.

### REFERENCES

1. Stryke, D., Kawamoto, M., Huang, C. C., Johns, S. J., King, L. A., Harper, C. A., Meng, E. C., Lee, R. E., Yee, A., L'Italien, L. et al. (2003) BayGenomics: a resource of insertional mutations in mouse embryonic stem cells. Nucleic Acids Res, 31, 278-281.
2. Skarnes, W. C., von Melchner, H., Wurst, W., Hicks, G., Nord, A. S., Cox, T., Young, S. G., Ruiz, P., Soriano, P., Tessier-Lavigne, M. et al. (2004) A public gene trap resource for mouse functional genomics. Nature Genetics, 36, 543-544.
3. Austin, C. P., Battey, J. F., Bradley, A., Bucan, M., Capecchi, M., Collins, F. S., Dove, W. F., Duyk, G., Dymecki, S., Eppig, J. T. et al. (2004) The knockout mouse project. Nat Genet, 36, 921-924.
4. Li, L. and Cohen, S. N. (1996) Tsg101: a novel tumor susceptibility gene isolated by controlled homozygous functional knockout of allelic loci in mammalian cells. Cell, 85, 319-329.
5. Hubbard, S. C., Walls, L., Ruley, H. E. and Muchmore, E. A. (1994) Generation of Chinese hamster ovary cell glycosylation mutants by retroviral insertional mutagenesis: integration into a discrete locus generates mutants expressing high levels of N-glycolyneuraminic acid. J. Biol. Chem., 269, 3717-3724.
6. Organ, E. L., Sheng, J., Ruley, H. E. and Rubin, D. H. (2004) Discovery of mammalian genes that participate in virus infection. BMC Cell Biol, 5, 41.
7. Guo, G., Wang, W. and Bradley, A. (2004) Mismatch repair genes identified using genetic screens in Bim-deficient embryonic stem cells. Nature, 429, 891-895.
8. Sheng, J., Organ, E. L., Hao, C., Wells, K. S., Ruley, H. E. and Rubin, D. H. (2004) Mutations in the IGF-II pathway that confer resistance to lytic reovirus infection. BMC Cell Biol, 5, 32.
9. Koike, H., Horne, K., Fukuyama, H., Kondoh, G., Nagata, S, and Takeda, J. (2002) Efficient biallelic mutagenesis with Cre/loxP-mediated inter-chromosomal recombination. EMBO Rep, 3, 433-437.
10. Liu, P., Jenkins, N. A. and Copeland, N. G. (2002) Efficient Cre-loxP-induced mitotic recombination in mouse embryonic stem cells. Nat Genet, 30, 66-72.
11. Morley, A. A. (1991) Mitotic recombination in mammalian cells in vivo. Mutat Res, 250, 345-349.
12. Shao, C., Deng, L., Henegariu, O., Liang, L., Raikwar, N., Sahota, A., Stambrook, P. J. and Tischfield, J. A. (1999) Mitotic recombination produces the majority of recessive fibroblast variants in heterozygous mice. Proc Natl Acad Sci USA, 96, 9230-9235.
13. Gupta, P. K., Sahota, A., Boyadjiev, S. A., Bye, S., Shao, C., O'Neill, J. P., Hunter, T. C., Albertini, R. J., Stambrook, P. J. and Tischfield, J. A. (1997) High frequency in vivo loss of heterozygosity is primarily a consequence of mitotic recombination. Cancer Res, 57, 1188-1193.
14. Wijnhoven, S. W., Kool, H. J., van Teijlingen, C. M., van Zeeland, A. A. and Vrieling, H. (2001) Loss of heterozygosity in somatic cells of the mouse. An important step in cancer initiation? Mutat Res, 473, 23-36.
15. Mortensen, R. M., Conner, D. A., Chao, S., Geisterfer-Lowrance, A. A. and Seidman, J. G. (1992) Production of homozygous mutant ES cells with a single targeting construct. Mol. Cell. Biol., 12, 2391-2395.
16. Paludan, K., Duch, M., Jorgensen, P., Kjeldgaard, N. O. and Pedersen, F. S. (1989) Graduated resistance to G418 leads to differential selection of cultured mammalian cells expressing the neo gene. Gene, 85, 421-426.
17. Lefebvre, L., Dionne, N., Karaskova, J., Squire, J. A. and Nagy, A. (2001) Selection for transgene homozygosity in embryonic stem cells results in extensive loss of heterozygosity. Nat Genet, 27, 257-258.
18. Ishida, Y. and Leder, P. (1999) RET: a poly A-trap retrovirus vector for reversible disruption and expression monitoring of genes in living cells. Nucleic Acids Res, 27, e35.
19. Yoshida, M., Yagi, T., Furuta, Y., Takayanagi, K., Kominami, R., Takeda, N., Tokunaga, T., Chiba, J., Ikawa, Y. and Aizawa, S. (1995) A new strategy of gene trapping in ES cells using 3′RACE. Transgenic Res, 4, 277-287.
20. Zambrowicz, B., Friedrich, G. A., Buxton, E. C., Lilleberg, S. L., Person, C. and Sands, A. T. (1998) Disruption and sequence identification of 2,000 genes in mouse embryonic stem cells. Nature, 392, 608-611.
21. Hardouin, N. and Nagy, A. (2000) Gene-trap-based target site for cre-mediated transgenic insertion. Genesis, 26, 245-252.
22. Araki, K., Imaizumi, T., Sekimoto, T., Yoshinobu, K., Yoshimuta, J., Akizuki, M., Miura, K., Araki, M. and Yamamura, K. (1999) Exchangeable gene trap using the Cre/mutated lox system. Cell Mol Biol (Noisy-le-grand), 45, 737-750.
23. Osipovich, A. B., Singh, A. and Ruley, H. E. (2005) Post-entrapment genome engineering: first exon size does not affect the expression of fusion transcripts generated by gene entrapment. Genome Res, 15, 428-435.
24. Cobellis, G., Nicolaus, G., Iovino, M., Romito, A., Marra, E., Barbarisi, M., Sardiello, M., Di Giorgio, F. P., Iovino, N., Zollo, M. et al. (2005) Tagging genes with cassette-exchange sites. Nucleic Acids Res, 33, e44.
25. Zheng, B., Sage, M., Cai, W. W., Thompson, D. M., Tavsanli, B. C., Cheah, Y. C. and Bradley, A. (1999) Engineering a mouse balancer chromosome. Nat Genet, 22, 375-378.
26. Ramirez-Solis, R., Liu, P. and Bradley, A. (1995) Chromosome engineering in mice. Nature, 378, 720-724.
27. Salminen, M., Meyer, B. I. and Gruss, P. (1998) Efficient poly A trap approach allows the capture of genes specifically active in differentiated embryonic stem cells and in mouse embryos. Dev Dyn, 212, 326-333.
28. Lee, G. and Saito, I. (1998) Role of nucleotide sequences of loxP spacer region in Cre-mediated recombination. Gene, 216, 55-65.
29. Osipovich, A. B., White-Grindley, E. K., Hicks, G. G., Roshon, M. J., Shaffer, C., Moore, J. H. and Ruley, H. E. (2004) Activation of cryptic 3′ splice sites within introns of cellular genes following gene entrapment. Nucleic Acids Res, 32, 2912-2924.
30. Cheng, J., Kapranov, P., Drenkow, J., Dike, S., Brubaker, S., Patel, S., Long, J., Stem, D., Tammana, H., Helt, G. et al. (2005) Transcriptional maps of 10 human chromosomes at 5-nucleotide resolution. Science, 308, 1149-1154.
31. Shigeoka, T., Kawaichi, M. and Ishida, Y. (2005) Suppression of nonsense-mediated mRNA decay permits unbiased gene trapping in mouse embryonic stem cells. Nucleic Acids Res, 33, e20.
32. Dattani, M. T., Martinez-Barbera, J. P., Thomas, P. Q., Brickman, J. M., Gupta, R., Martensson, I. L., Toresson, H., Fox, M., Wales, J. K., Hindmarsh, P. C. et al. (1998) Mutations in the homeobox gene HESX1/Hesx1 associated with septo-optic dysplasia in human and mouse. Nat Genet, 19, 125-133.
33. Paupe, V., Gilbert, T., Le Merrer, M., Munnich, A., Cormier-Daire, V. and El Ghouzzi, V. (2004) Recent advances in Dyggve-Melchior-Clausen syndrome. Mol Genet Metab, 83, 51-59.
34. Wijnhoven, S. W., Sonneveld, E., Kool, H. J., van Teijlingen, C. M. and Vrieling, H. (2003) Chemical carcinogens induce varying patterns of LOH in mouse T-lymphocytes. Carcinogenesis, 24, 139-144.
35. Cao, S., Bendall, H., Hicks, G. G., Nashabi, A., Sakano, H., Shinkai, Y., Gariglio, M., Oltz, E. M. and Ruley, H. E. (2003) The high-mobility-group box protein SSRP1/T160 is essential for cell viability in day 3.5 mouse embryos. Mol Cell Biol, 23, 5301-5307.
36. Vainberg, I. E., Lewis, S. A., Rommelaere, H., Ampe, C., Vandekerckhove, J., Klein, H. L. and Cowan, N. J. (1998) Prefoldin, a chaperone that delivers unfolded proteins to cytosolic chaperonin. Cell, 93, 863-873.
37. Stoler, D. L., Chen, N., Basik, M., Kahlenberg, M. S., Rodriguez-Bigas, M. A., Petrelli, N. J. and Anderson, G. R. (1999) The onset and extent of genomic instability in sporadic colorectal tumor progression. Proc Natl Acad Sci USA, 96, 15121-15126.
38. Yusa, K., Horie, K., Kondoh, G., Kouno, M., Maeda, Y., Kinoshita, T. and Takeda, J. (2004) Genome-wide phenotype analysis in ES cells by regulated disruption of Bloom's syndrome gene. Nature, 429, 896-899.
39. Hicks, G. G., Shi, E.-G., Li, X.-M., Li, C.-H., Pawlak, M. and Ruley, H. E. (1997) Functional genomics in mice by tagged sequence mutagenesis. Nature Genetics, 16, 338-344.
40. von Melchner, H., DeGregori, J. V., Rayburn, H., Reddy, S., Friedel, C. and Ruley, H. E. (1992) Selective disruption of genes expressed in totipotent embryonal stem cells. Genes Dev., 6, 919-927.
41. Wu, X., Li, Y., Crise, B. and Burgess, S. M. (2003) Transcription start regions in the human genome are favored targets for MLV integration. Science, 300, 1749-1751.
42. Nowell, P. C. (1976) Science 194, 23-28.
43. Ponder, B. A. (2001) Nature 411, 336-341.
44. Ames, B. N., Gold, L. S. & Willett, W. C. (1995) Proc Natl Acad Sci USA 92, 5258-5265.
45. Peto, J. (2001) Nature 411, 390-395.
46. Wogan, G. N., Hecht, S. S., Felton, J. S., Conney, A. H. & Loeb, L. A. (2004) Semin Cancer Biol 14, 473-486.
47. Lengauer, C., Kinzler, K. W. & Vogelstein, B. (1998) Nature 396, 643-649.
48. Loeb, L. A., Loeb, K. R. & Anderson, J. P. (2003) Proc Natl Acad Sci USA 100, 776-781.
49. Rajagopalan, H. & Lengauer, C. (2004) Nature 432, 338-341.
50. Hoeijmakers, J. H. (2001) Nature 411, 366-374.
51. Nyberg, K. A., Michelson, R. J., Putnam, C. W. & Weinert, T. A. (2002) Annu Rev Genet. 36, 617-656.
52. Barnes, D. E. & Lindahl, T. (2004) Annu Rev Genet. 38, 445-476.
53. Risinger, M. A. & Groden, J. (2004) Cancer Cell 6, 539-545.
54. Weaver, B. A. & Cleveland, D. W. (2005) Cancer Cell 8, 7-12.
55. Boland, C. R. & Ricciardiello, L. (1999) Proc Natl Acad Sci USA 96, 14675-14677.
56. Schneider, B. L. & Kulesz-Martin, M. (2004) Carcinogenesis 25, 2033-2044.
57. Duesberg, P., Fabarius, A. & Hehlmann, R. (2004) IUBMB Life 56, 65-81.
58. Marx, J. (2002) Science 297, 544-546.
59. Shih, I. M., Zhou, W., Goodman, S, N., Lengauer, C., Kinzler, K. W. & Vogelstein, B. (2001) Cancer Res 61, 818-822.
60. Luo, L., Li, B. & Pretlow, T. P. (2003) Cancer Res 63, 6166-6169.
61. Chen, R., Rabinovitch, P. S., Crispin, D. A., Emond, M. J., Koprowicz, K. M., Bronner, M. P. & Brentnall, T. A. (2003) Am J Pathol 162, 665-672.
62. Feinberg, A. P. (2004) Semin Cancer Biol 14, 427-432.
63. Futreal, P. A., Coin, L., Marshall, M., Down, T., Hubbard, T., Wooster, R., Rahman, N. & Stratton, M. R. (2004) Nat Rev Cancer 4, 177-183.
64. Tomlinson, I., Sasieni, P. & Bodmer, W. (2002) Am J Pathol 160, 755-758.
65. Cairns, J. (2002) Proc Natl Acad Sci USA 99, 10567-10570.
66. Cervantes, R. B., Stringer, J. R., Shao, C., Tischfield, J. A. & Stambrook, P. J. (2002) Proc Natl Acad Sci USA 99, 3586-3590.
67. Saretzki, G., Armstrong, L., Leake, A., Lako, M. & von Zglinicki, T. (2004) Stem Cells 22, 962-971.
68. Hong, Y. & Stambrook, P. J. (2004) Proc Natl Acad Sci USA 101, 14443-14448.
69. Aladjem, M. I., Spike, B. T., Rodewald, L. W., Hope, T. J., Klemm, M., Jaenisch, R. & Wahl, G. M. (1998) Curr Biol 8, 145-155.
70. Luo, G., Santoro, I. M., McDaniel, L. D., Nishijima, I., Mills, M., Youssoufian, H., Vogel, H., Schultz, R. A. & Bradley, A. (2000) Nat Genet. 26, 424-429.
71. Reya, T., Morrison, S. J., Clarke, M. F. & Weissman, I. L. (2001) Nature 414, 105-111.
72. Hanks, S., Coleman, K., Reid, S., Plaja, A., Firth, H., Fitzpatrick, D., Kidd, A., Mehes, K., Nash, R., Robin, N., Shannon, N., Tolmie, J., Swansbury, J., Irrthum, A., Douglas, J. & Rahman, N. (2004) Nat Genet. 36, 1159-1161.
73. Mazur-Melnyk, M., Stuart, G. R. & Glickman, B. W. (1996) Mutat Res 358, 89-96.
74. Vogel, E. W. & Nivard, M. J. (1993) Mutagenesis 8, 57-81.
75. Wijnhoven, S. W., Van Sloun, P. P., Kool, H. J., Weeda, G., Slater, R., Lohman, P. H., van Zeeland, A. A. & Vrieling, H. (1998) Proc Natl Acad Sci USA 95, 13759-13764.
76. Stettler, P. M. & Sengstag, C. (2001) Mol Carcinog 31, 125-138.
77. Chen, T., Harrington-Brock, K. & Moore, M. M. (2002) Mutagenesis 17, 105-109.
78. Turner, D. R., Dreimanis, M., Holt, D., Firgaira, F. A. & Morley, A. A. (2003) Mutat Res 522, 21-26.
79. Wang, T. L., Rago, C., Silliman, N., Ptak, J., Markowitz, S., Willson, J. K., Parmigiani, G., Kinzler, K. W., Vogelstein, B. & Velculescu, V. E. (2002) Proc Natl Acad Sci U S A 99, 3076-3080.

1. A retroviral poly(A) trap vector comprising a nucleotide sequence between a 5′ LTR and a 3′ LTR, wherein said nucleotide sequence comprises 1) an intron containing nucleic acid encoding a first selective marker operably linked to a promoter, 2) site specific recombinase sites, and 3) a 3′ exon comprising a nucleic acid encoding the 3′ segment of a second selective marker, an internal ribosome entry site (IRES), a nucleic acid encoding a reporter protein and a polyadenylation site.

2. The vector of claim 1, wherein the first selective marker is neomycin.

3. The vector of claim 1 wherein the second selective marker is puromycin.

4. The vector of claim 1 wherein the promoter is the RNA polymerase 2 promoter.

5. The vector of claim 1, wherein the vector is the vector shown in FIG. 1A.

6. A method of selecting cells with homozygous mutations in their genomes comprising:
a) contacting cells with the vector of claim 1;
b) selecting cells with mutations induced by insertion of the vector into a cellular gene;
c) exposing the cells to conditions that select for cells homozygous for vector induced mutations;
d) selecting cells that survive under the selective condition of step c).

7. A method of producing cells with increased frequency of homozygous mutations in their genomes comprising:
a) contacting cells with the vector of claim 1;
b) exposing the cells to a carcinogen;
c) exposing the cells to condition(s) that select for cells homozygous for vector induced mutations
d) selecting cells that survive under the selective condition(s) of step c).

8-21. (canceled)

22. A method of identifying a gene necessary for infection and nonessential for cellular survival comprising:
a) contacting cells with the vector of claim 1;
b) contacting the cells with a pathogen;
c) selecting cells that survive and exhibit resistance to infection in the absence of gene function; and
d) identifying the cellular gene disrupted by the vector.

23. The method of claim 22, wherein the pathogen is a virus, a bacterium or a parasite.

24-30. (canceled)
